(19) United States
(12) Patent Application Publication
(10) Pub. No.: US 2009/0215713 A1
(43) Pub. Date: Aug. 27, 2009

(54) Use of atp for the manufacture of a medicament for the prevention and treatment of oxidative stress and related conditions

(75) Inventors: Pieter C. Dagnelie, Maastricht (NL); Els L.R. Swennen, Maastricht (NL); Aalt Bast, Maastricht (NL); Arno T.P. Skrabanja, Maastricht (NL); Sandra Beijer, Maastricht (NL); Martijin J.I. Bours, Maastricht (NL)

Correspondence Address: KALOW & SPRINGUT LLP, 488 MADISON AVENUE, 19TH FLOOR, NEW YORK, NY, 10022, US

(73) Assignee: UNIVERSITEIT VAN MAASTRICHT, Maastricht (NL)

(21) Appl. No.: 11/597,668

(22) PCT Filed: May 23, 2005
(86) PCT No.: PCT/EP05/05652

§ 371 Date: Dec. 4, 2008

(30) Foreign Application Priority Data
May 23, 2004 (EP) 04076502.6

(51) Int. Cl. A61K 31/7076; C07H 19/20; A61P 1/00

(52) U.S. Cl. 514/47; 536/26.26

(57) ABSTRACT

The present invention provides the use of ATP for the manufacture of a medicine comprising ATP as an active ingredient for exerting a preventive or therapeutic pharmacological effect when administered to a mammal, preferably a human, selected from the group consisting of: a. modulating oxidative stress and the effects thereof by favourably affecting the formation or scavenging of aggressive hydroxyl radicals; b. modulating the inflammatory response to a strong external insult such as endotoxin (LPS) and/or phytohaemagglutinin, even under conditions of severe oxidative stress; c. inhibiting the inflammatory response to a strong external insult such as endotoxin (LPS) and/or phytohaemagglutinin under conditions of severe oxidative stress; d. exerting a local protective effect against oxidative stress in the intestine, thus preventing intestinal damage induced by several types of medication such as non steroid anti-inflammatory drugs (NSAIDs); e. exerting favourable immuno-modulating and oxidative stress-reducing effects in blood from patients with oxidative stress-related disorders; and f. exerting favourable clinical effects in patients with different oxidative stress-related disorders such as, but not limited to, rheumatoid arthritis, intestinal disease, cancer and fatigue. The medicine is preferably manufactured in lyophilized form.

### FIELD OF THE INVENTION

The present invention relates to the use of adenosine 5′-triphosphate in the prevention and treatment of conditions which are caused or accompanied by increased oxidative stress due to excessive formation of reactive oxygen species by any cause, including conditions of aberrant, excessive, depressed, or insufficient immune response and fatigue in mammals, in particular humans. Furthermore, the invention relates to a novel pharmaceutical composition comprising ATP and to a dedicated infusion device for intravenous administration of ATP, which combination greatly facilitates safe and subject-friendly ATP administration in a non-medical setting, such as in private homes, nursing homes, and the like.

### BACKGROUND OF THE INVENTION

a. Prior Art Relating to ATP and its Applications in General

Adenosine 5′-triphosphate (ATP) is a naturally occurring nucleotide which is present in every cell. Nucleotides were first recognised as important substrate molecules in metabolic interconversions, and later as the building blocks of DNA and RNA. More recently, it was found that nucleotides are also present in the extracellular fluid under physiologic circumstances. The prior art concerning the physiology and established and potential clinical applications of ATP, as well as its pharmacokinetic properties, physiological effects and mechanisms of action has been reviewed (1).

ATP has recently aroused interest because of its properties as a signaling substance outside the cell (extracellular ATP). Extracellular ATP is known to be involved in the regulation of a variety of biological processes including neurotransmission, muscle contraction, cardiac function, platelet function, and vasodilatation.

ATP can be released from the cytoplasm of several cell types and interacts with specific purinergic receptors, which are present on the surface of many cells and play a fundamental role in cell physiology. Intravenous administration of ATP induces a rapid rise in ATP levels uptake by erythrocytes (2) and liver (3) followed by slow release into the plasma compartment.

In the past years, possible pharmacological uses of ATP have received attention, following reports of its potential benefit in pain, vascular diseases and cancer. ATP has cytostatic and cytotoxic effects in many types of transformed and tumour cells (for review, see (1)).

In vivo daily intraperitoneal injections of 25 mmol/L ATP, AMP or adenosine for 10 consecutive days into mice bearing colon tumour induced a significant inhibition of host weight loss in this experimental cancer model (4). This inhibition was associated with expansion of erythrocyte ATP pools (5).

In the USA, a phase I/II trial was carried out in 8 stage IIIB/IV patients with non-small cell lung cancer. After treatment with 2 to 3 intravenous ATP courses of 96 hours at 4-week intervals, stabilisation of body weight was observed (6). In a subsequent open-ended phase II trial in 15 newly diagnosed patients with non-small cell lung cancer, an average weight gain of 1.3 kg was demonstrated after 4 ATP courses (7).

In a randomized clinical trial in advanced non-small-cell lung cancer patients (8), it was shown that regular infusions of adenosine 5′-triphosphate (ATP) inhibited loss of weight and muscle mass compared to a control group of non-small-cell lung cancer patients (stage IIIB or IV) receiving usual non-small cell lung cancer supportive care only. Moreover, physical and functional quality of life, appetite, and muscle strength remained stable in the ATP group, but progressively deteriorated in the control group. Although preliminary data from a small subset of cancer patients suggested potential inhibition of C-reactive protein by ATP, further analyses showed neither an effect of ATP on blood sedimentation rate (Dagnelie, unpublished data 2005), nor on plasma levels of pro- or anti-inflammatory cytokines in this patient population (Swennen et al. 2004).

In all studies published to date, ATP was administered at maximum doses of 75-100 μg/kg·min over periods of 24-96 h. The prevailing view as published in the literature is that, generally speaking, administration of a relatively high infusion dose of ATP, approximating the above maximum dose of 75-100 μg/kg·min, is preferred because it is expected to have a greater efficacy than lower doses of ATP.

b. Patent Literature

The patent literature also reveals a variety of applications and developments relating to adenosine triphosphate (ATP) and other adenosine derivatives including adenosine.

For example, EP 0 352 477 of Rapaport discloses the use of AMP, ADP and ATP in the treatment of cancer-related cachexia.

U.S. Pat. No. 4,880,918 and U.S. Pat. No. 5,049,372 to Rapaport disclose anticancer activities (i.e. inhibition of the growth of tumor cells) in a host by increasing blood and plasma ATP levels.

U.S. Pat. No. 5,227,371 to Rapaport discloses the administration of AMP, ATP or their degradation products adenosine and inorganic phosphate to a host, achieving the beneficial increases in ATP levels in liver, total blood and blood plasma.

U.S. Pat. No. 5,547,942 to Rapaport discloses the administration of ATP or other adenine nucleotides and inorganic phosphates to human patients in treating non-insulin-dependent diabetes mellitus following the interactions of extracellular ATP pools with pancreatic beta cell purine receptors.

U.S. Pat. No. 6,159,942 to St. Cyr et at discloses the oral administration of precursors of ATP, in particular pentose sugars such as D-ribose, to increase intracellular ATP concentration as dietary supplements or for treatment of reduced energy availability resulting from strenuous physical activity, illness or trauma.

US 2003/0109486 to Rapaport discloses methods for the utilization of ATP in the treatment of acute lung injury (ALI) and acute respiratory distress syndrome (ARDS).

WO 01/028528 of Rapaport discloses methods for preventing/reducing weight gain by administering ATP in coated form for the chronic administration of adenosine, aiming at desentisizing A1 adenosine receptors towards the action of adenosine and thereby increasing intracelallur levels of cyclic AMP, thereby resulting in stimulation of lipolysis.

US 2003/0069203 to Lee et al. discloses a composition for oral administration used for improving muscle torque and reducing muscle fatigue comprising an effective amount of ATP in an enteric coating that protects ATP from degradation by gastric juices, to enhance absorption into the blood stream and provide additional therapeutic benefit.

WO 03/039473 of Peterson and Yerxa discloses a composition for treating dry eye disease. Although an effect of ATP in other inflammatory conditions is also claimed, no empirical support for this statement has been provided whatsoever.

WO 03/061568 of Rapaport discloses that continuous intravenous infusions of ATP at a maximum rate as high as 100 μg/kg·min are administered. It is also mentioned that ATP is administered for a minimum of 8 weekly cycles.

c. Prior Knowledge Regarding Intravenous ATP Administration in Humans

In all reports published to date, intravenous ATP administration was performed under strict medical supervision, either at a medical ward or in a day care center of the hospital, because of the adherent risk of potential side effects of ATP. However, there are several major limitations to the application of ATP administered in such a hospital setting:
The regular stays at the hospital ward or day care center for ATP infusions (e.g. once per 1-4 weeks) comprise a considerable burden to patients,
These ATP infusions put a high demand on scarce resources of hospital beds and specialized medical care,
And cause high costs for the health care system,

For reasons of patients' safety, there has been no attempt to administer ATP outside a strict medical setting to date. In particular, WO 03/061568 (Rapaport) discloses the administration of ATP over a period of typically 8-10 hours in an outpatient setting within the hospital. The patent specification is allegedly based on the observation that short, weekly, continuous infusions of ATP, “at infusion rates even somewhat higher than what has been previously reported”, resulted in similar clinical efficacies with significantly reduced profiles of adverse effects compared to longer (30-96 hrs) infusions. However, our experience with over 200 ATP infusions varying in dose (25-75 μg/kg·min) and duration (8-30 hrs) demonstrates that side effects induced by intravenous ATP infusion only depend on the infusion rate, and not on the duration of the ATP infusions. Moreover, in contrast with quotations of our work in the aforementioned patent application, we did not find any life-threatening side effects in our previous study with ATP infusion during 30 hrs (8), as was correctly quoted in Rapaport's US 2003/0109486. Thus, both our data and US 2003/0109486 contradict the disclosure of WO 03/061568.

There is a continuous interest in exploring possible further pharmacological uses of ATP and ways of administering ATP because of its favourable properties hitherto known combined with the favourable safety profile of ATP. The present invention provides new uses of this substance with promising results, as well as novel ways and methods for facilitating the administration of ATP without direct medical supervision, e.g. at private homes, nursing homes, etc.

### SUMMARY OF THE INVENTION

It has now been surprisingly found, after extensive research and testing, that ATP: 1°. favourably affects hydroxyl radical formation or scavenging from H₂O₂ during Fenton chemistry, i.e. ATP and its analogues inhibit the formation of the spin adduct DMPO—OH in electron spin resonance (ESR) experiments; 2°. by virtue of the effect mentioned under 1°, markedly inhibits the inflammatory response to an insult inducing severe oxidative stress, such as H₂O₂ or γ-irradiation; 3°. inhibits the inflammatory response to a strong external insult such as endotoxin (LPS) and/or phytohaemagglutinin under conditions of severe oxidative stress; 4°. exerts a local oxidative stress and intestinal permeability attenuating effect in the intestine, thus preventing intestinal damage induced by several types of medication including so-called non-steroid anti-inflammatory drugs (NSAIDs); 5°. exerts favourable immuno-modulating and oxidative stress-reducing effects in blood from patients with different oxidative stress-related disorders, as described in the Experimental Section; and 6°. exerts favourable clinical effects in patients with different oxidative stress-related disorders, such as rheumatoid arthritis, cancer chronic fatigue, and the like.

Therefore, in a first aspect the present invention provides the use of ATP for the manufacture of a medicine comprising ATP as an active ingredient for exerting a preventive or therapeutic pharmacological effect when administered to a mammal, preferably a human, selected from the group consisting of:
a. modulating oxidative stress and the effects thereof by favourably affecting the formation or scavenging of aggressive hydroxyl radicals;
b. modulating the inflammatory response to a strong external insult such as endotoxin (LPS) and/or phytohaemagglutinin, even under conditions of severe oxidative stress;
c. inhibiting the inflammatory response to a strong external insult such as endotoxin (LPS) and/or phytohaemagglutinin under conditions of severe oxidative stress;
d. exerting a local protective effect against oxidative stress in the intestine, thus preventing intestinal damage induced by several types of medication such as non-steroid anti-inflammatory drugs (NSAIDs);
e. exerting favourable immuno-modulating and oxidative stress-reducing effects in blood from patients with oxidative stress-related disorders; and
f. exerting favourable clinical effects in patients with different oxidative stress-related disorders such as, but not limited to, rheumatoid arthritis, intestinal disease, cancer and fatigue.

In a further aspect of the present invention, the use of ATP is provided for the manufacture of a medicine comprising ATP as an active ingredient having a preventive or curative activity when administered to a mammal, preferably a human, selected from the group consisting of:
g. tissue-protecting activity by attenuating oxidative stress under varying conditions of oxidative stress and inflammation;
h. immune-stimulating activity by attenuating oxidative stress under varying conditions characterized by immune-incompetence or immuno-suppression, and immuno-modulating activity normalizing the Th1/Th2 balance in conditions of aberrant Th1- or Th2-skewed immune response, such as auto-immune disorders and atopic diseases; and
i. modulating and normalizing aberrant mental neurological and neuro-psychiatric states and diseases.

In still a further aspect of the present invention the use of ATP is provided for the manufacture of a medicine comprising ATP as an active ingredient wherein the medicine is for preventing or treating at least one of intestinal inflammatory condition, intestinal damage, and inflammatory bowel disease.

In yet another aspect of the present invention the use of ATP is provided for the manufacture of a medicine comprising ATP as an active ingredient wherein the medicine is for preventing or treating rheumatoid arthritis.

In a further aspect of the present invention the use of ATP is provided for the manufacture of a medicine comprising ATP as an active ingredient wherein the medicine is for preventing or treating atopic disease, including asthma.

In another aspect of the present invention the use of ATP is provided for the manufacture of a medicine comprising ATP as an active ingredient wherein the medicine is for preventing or treating a condition selected from the group consisting of fatigue, fibromyalgia, burn-out and depression.

In still a further aspect of the present invention the use of ATP is provided for the manufacture of a medicine comprising ATP as an active ingredient wherein the medicine is for preventing or treating an individual for a disease or disorder or condition selected from the group consisting of intestinal inflammation, intestinal damage, rheumatoid arthritis, COPD, cancer during or after treatment by at least one of surgery, radiotherapy, and chemotherapy, a neurological or mental disorder, an atopic disease including asthma, and another condition of elevated or aberrant inflammatory response, for example an auto-immune disorder, disease and condition of immunosuppression, immuno-incompetence and limited resistance towards infections, such as caused by disease or agents, for example human immunodeficiency virus (HIV) or acquired immune deficiency syndrome (AIDS), or limited resistance towards infections.

In yet another aspect of the invention a method is provided of preventing or treating an individual for a disease or disorder or condition selected from the group consisting of intestinal inflammation, intestinal damage, rheumatoid arthritis, COPD, cancer during or after treatment by at least one of surgery, radiotherapy, and chemotherapy, a neurological or mental disorder, an atopic disease including asthma, and another condition of elevated or aberrant inflammatory response, which comprises administering to said individual in need thereof a medicine comprising an effective amount of ATP.

Furthermore, it has been surprisingly found that the effective dose of ATP is considerably lower than was hitherto thought and as compared with the prior art. We have found that in rheumatoid arthritis, ATP was highly effective in improving disease symptoms within 4 courses at a dose of 10-15 μg/kg·min. In pre-terminal cancer patients, improved self-reliance was seen at a dose of 30 μg/kg·min. In patients with chronic fatigue syndrome, the majority of patients received effective ATP infusions at a rate ≦40 μg/kg·min. Thus, in a further aspect of the invention pharmaceutical compositions are provided comprising ATP as an active ingredient in a dose form preferably ranging as low as 5-40 μg/kg·min, more preferably 10-30 μg/kg·min, especially 10-20 μg/kg·min.

In a preferred embodiment of the invention the medicine is in the form of a pharmaceutical composition or a nutritional composition, and is most preferably in a lyophilized form, preferably in conjunction with a suitable adjuvant, such as mannitol. Prior to administration to an individual, a lyophilized ATP composition is preferably dissolved in a suitable solvent, such as PBS, for example by injection or infusion. In a preferred way of application, the medicine is administered using a special device including a dedicated infusion pump.

These and other aspects of the invention will be discussed below in more detail.

### BRIEF DESCRIPTION OF THE DRAWINGS

FIG. 1 Effect of ATP on the formation or scavenging of hydroxyl radicals during Fenton chemistry: representative electron spin resonance (ESR) spectra. Panel A shows the control ESR spectra (buffer), and panel B shows ESR spectra in the presence of ATP. The effect of ATP on the formation or scavenging of hydroxyl radicals, generated by Fenton reagents was measured as DMPO—OH spin adducts in ESR spectra. The ESR studies were performed at room temperature using a Bruker EMX 1273 spectrometer equipped with an ER 4119HS high sensitivity cavity and 12 kW power supply. The following instrument conditions were used: scan range, 60 G; center magnetic field, 3490 G; modulation amplitude, 1.0 G; microwave frequency, 9.86 GHz; time constant, 40.96 ms, scan time, 20.48 ms; number of scans, 50. OH radicals were generated by the Fenton reaction, and 5,5-dimethyl-1-pyrroline N-oxide (DMPO) was used for trapping hydroxyl radicals. Fifty microliters of 10 mM H₂O₂, 50 μl of 250 mM DMPO, 50 μl milliQ, 50 μl milliQ (control) or sample, and 50 μl 5 mM FeSO₄/5 mM EDTA were mixed, and then transferred to a capillary glass tube. After 2 minutes, DMPO—OH spin adducts were measured by ESR. Quantification of the spectra was performed by peak integration using the WIN-EPR spectrum manipulation program. Results showed that ATP strongly inhibited the formation of the DMPO—OH spin adduct during Fenton chemistry.

FIG. 2 Effect of ATP on the formation or scavenging of hydroxyl radicals during Fenton chemistry. After addition of ATP (final concentration 0.1 mM-10 mM), DMPO—OH spin adducts were quantified using electron spin resonance (ESR) spectrometry. Presented values are means of triplicate determinations, 100% being the percent hydroxyl radicals when no ATP is present (control). Results showed that ATP prevents the formation of the DMPO—OH spin adduct during Fenton chemistry in a concentration-dependent manner, with ≈80% inhibition of the DMPO—OH spin adduct formation at the highest ATP concentration. The effect was statistically significant (P<0.05) at all ATP concentrations between 0.1 and 10 mM.

FIG. 3 Effect of ADP on the formation or scavenging of hydroxyl radicals during Fenton chemistry. After addition of ADP (final concentration 0.1 mM-10 mM), DMPO—OH spin adducts were quantified using ESR spectrometry. Presented values are means of triplicate determinations, 100% being the percent hydroxyl radicals when no ADP is present (control). Results showed that ADP inhibited the formation of the DMPO—OH spin adduct during Fenton chemistry in a concentration-dependent manner, with ≈70% inhibition at the highest ADP concentration. The effect was statistically significant (P<0.05) at ADP concentrations between 0.3 mM and 10 mM.

FIG. 4 Effect of AMP on the formation or scavenging of hydroxyl radicals during Fenton chemistry. After addition of AMP (final concentration 0.1 mM-10 mM), DMPO—OH spin adducts were quantified using ESR spectrometry. Presented values are means of triplicate determinations, 100% being the percent hydroxyl radicals when no AMP is present (control). Results showed that, only at concentrations of 3 and 10 mM, AMP induced a significant (P<0.05) reduction in hydroxyl radicals; however, no significant effect on hydroxyl radicals was found at lower concentrations (0.1, 0.3 and 1 mM).

FIG. 5 Effect of adenosine on the formation or scavenging of hydroxyl radicals. After addition of adenine (final concentration 1 mM), DMPO—OH spin adducts were quantified using ESR spectrometry. Presented values are means of triplicate determinations, 100% being the percent hydroxyl radicals when no adenosine is present (control). Results showed that adenosine at the concentration of 1 mM had no significant (P<0.05) effect on the formation of the DMPO—OH spin adduct during Fenton chemistry.

FIG. 6 Effect of adenine on the formation or scavenging of hydroxyl radicals. After addition of adenine (final concentration 1 mM), DMPO—OH spin adducts were quantified using ESR spectrometry. Presented values are means of triplicate determinations, 100% being the percent hydroxyl radicals when no adenine is present (control). Results showed that adenine at the concentration of 1 mM had no significant (P<0.05) effect on the formation of the DMPO—OH spin adduct during Fenton chemistry.

FIG. 7 Effect of ATP on LPS+PHA-induced TNF-α secretion in whole blood from healthy subjects. The whole blood was exposed to 10 μg/ml LPS and 1 μg/ml PHA with indicated concentrations of ATP for 24 h. The TNF-α released into the supernatants was analyzed using the ELISA method. Results are expressed in percentage, 100% being the TNF-α release under stimulation by LPS+PHA without ATP. The TNF-α release induced by LPS+PHA from whole blood was significantly inhibited by the addition of ATP. Data are expressed as the mean values; error bars represent SEM. *, different from control (stimulation by LPS+PHA without ATP) (P<0.05).

FIG. 8 Effect of ATP on LPS+PHA-induced IL-10 secretion in whole blood from healthy subjects. The whole blood was exposed to 10 μg/ml LPS and 1 μg/ml PHA with indicated concentrations of ATP for 24 h. The IL-10 released into the supernatants was analyzed using the ELISA method. Results are expressed in percentage, 100% being the IL-10 release under stimulation by LPS+PHA without ATP. The IL-10 release induced by LPS+PHA from whole blood was significantly increased by the addition of ATP. Data are expressed as the mean values; error bars represent SEM. *, different from control (stimulation by LPS+PHA without ATP) (P<0.05).

FIG. 9 Effect of ATP on LPS+PHA-induced IL-6 secretion in whole blood from healthy subjects. The whole blood was exposed to 10 μg/ml LPS and 1 μg/ml PHA with indicated concentrations of ATP for 24 h. The IL-6 released into the supernatants was analyzed using the ELISA method. Results are expressed in percentage, 100% being the IL-6 release under stimulation by LPS+PHA without ATP. The IL-6 release induced by LPS+PHA from whole blood was not influenced by the addition of ATP. Data are expressed at the mean values; error bars represent SEM.

FIG. 10 Effect of ATP on LPS+PHA-induced TNF-α secretion in whole blood from healthy subjects under conditions of oxidative stress. Two concentrations of H₂O₂ (1 and 10 mM) were added to whole blood, followed by the incubation with the concentrations of ATP. Then, blood was exposed to 10 μg/ml LPS and 1 μg/ml PHA, and incubated for 24 h. The TNF-α released into the supernatants was analyzed using the ELISA method. The TNF-α release induced by LPS+PHA from whole blood was significantly inhibited by the addition of ATP. Data are expressed as the mean values; error bars represent SEM.

FIG. 11 Effect of ATP on LPS+PHA-induced IL-10 secretion in whole blood from healthy subjects under conditions of oxidative stress. Two concentrations of H₂O₂ (1 and 10 mM) were added to whole blood, together with the indicated concentrations of ATP. Then, blood was exposed to 10 μg/ml LPS and 1 μg/ml PHA, and incubated for 24 h. The IL-10 released into the supernatants was analyzed using the ELISA method. The IL-10 release induced by LPS+PHA from whole blood was significantly increased by the addition of ATP. Data are expressed as the mean values; error bars represent SEM.

FIG. 12 Effect of ATP on LPS+PHA-induced IL-6 secretion in whole blood from healthy subjects under conditions of oxidative stress. Two concentrations of H₂O₂ (1 and 10 mM) were added to whole blood, together with the indicated concentrations of ATP. Then, blood was exposed to 10 μg/ml LPS and 1 μg/ml PHA, and incubated for 24 h. The IL-6 released into the supernatants was analyzed using the ELISA method. The IL-6 release induced by LPS+PHA from whole blood was not influenced by the addition of ATP. Data are expressed at the mean values; error bars represent SEM.

FIG. 13 Effect of different purinergic compounds on LPS+PHA-induced TNF-α secretion in whole blood. The whole blood was exposed to 10 μg/ml LPS and 1 μg/ml PHA with the indicated purinergic compounds at the concentration of 300 μM for 24 h. The TNF-α released into the supernatant was analyzed using the ELISA method. Results are expressed in percentage, 100% being the TNF-α release under stimulation by LPS+PHA without addition of a purinergic compound (control). The TNF-α release induced by LPS+PHA from whole blood was inhibited by different compounds in the following order: adenosine (least inhibition)<AMP<ADP<ATP (greatest inhibition). The TNF-α release induced by LPS+PHA from whole blood was not inhibited by UTP, UDP or CTP. Data are expressed as the mean values; error bars represent SEM.

FIG. 14 Effect of ATP on cytokine secretion in whole blood under conditions of oxidative stress. Blood was pre-incubated with ATP or no ATP (control) for 30 min, followed by incubation with H₂O₂ (5 mM) or 24 h, without addition of LPS+PHA. The TNF-α and IL-10 released into the supernatant was analyzed using the ELISA method. Results are expressed in percentage, 100% being the TNF-α/IL-10 ratio when blood was incubated with H₂O₂ only, but no ATP (control). The TNF-alpha/IL-10 ratio was reduced by ATP in a concentration-dependent manner, with a 90% reduction at 300 μM ATP, indicating inhibition of inflammatory response.

FIG. 15 Effect of ATP on the ratio of TNF-α/IL-10 secretion in whole blood from a patient with oxidative stress-related disease. Panel (A) shows the effect of ATP on the TNF-α/IL-10 ratio in untreated whole blood, i.e. without LPS+PHA stimulation. Panel (B) shows the results in whole blood which was exposed to 10 μg/ml LPS and 1 μg/ml PHA with indicated concentrations of ATP for 24 h. In both experiments, TNF-α and IL-10 released into the supernatants was analyzed using the ELISA method. Results are expressed in percentage, 100% being the TNF-α/IL-10 ratio without ATP (control). Both in untreated and in LPS+PHA-stimulated blood, the ratio of TNF-α/IL-10 release from whole blood was markedly reduced by the addition of ATP. These results indicate that the effect of ATP in blood from healthy subjects is reproducible in patients with oxidative stress-associated disease, thus corroborating the practical relevance of the utilized blood model.

FIG. 16 Effect of ATP on the ratio of TNF-α/IL-10 secretion in whole blood from a patient with oxidative stress-related disease. Panel (A) shows the effect of ATP on the TNF-α/IL-10 ratio in untreated whole blood, i.e. without LPS+PHA stimulation. Panel (B) shows the results in whole blood which was exposed to 10 μg/ml LPS and 1 μg/ml PHA with indicated concentrations of ATP for 24 h. In both experiments, TNF-α and IL-10 released into the supernatants was analyzed using the ELISA method. Results are expressed in percentage, 100% being the TNF-α/IL-10 ratio without ATP (control). Both in untreated and in LPS-PHA stimulated blood, the ratio of TNF-α/IL-10 release from whole blood was reduced by the addition of ATP. These results indicate that the effect of ATP in blood from healthy subjects is reproducible in patients with oxidative stress-associated disease.

FIG. 17 Effect of ATP on the ratio of TNF-α/IL-10 secretion in whole blood from a patient with oxidative stress-related disease. This figure shows the results in whole blood which was exposed to 10 μg/ml LPS and 1 μg/ml PHA with indicated concentrations of ATP for 24 h. TNF-α and IL-10 released into the supernatants was analyzed using the ELISA method. Results are expressed in percentage, 100% being the TNF-α/IL-10 ratio without ATP (control). The ratio of TNF-α/IL-10 release from whole blood was reduced by the addition of ATP. These results indicate that the effect of ATP in blood from healthy subjects is reproducible in patients with oxidative stress-associated disease.

FIG. 18 Effect of ATP on cytokine secretion in whole blood after γ-irradiation. Blood was pre-incubated with 300 μM ATP or medium (control) for 30 minutes and then irradiated with γ-radiation (16 Gy). Results showed a marked irradiation-induced TNFα stimulation at 3 and 5 h post-irradiation. This TNFα stimulation was completely blocked by ATP, indicating inhibition of inflammatory response.

FIG. 19 Generation of reactive oxygen species (ROS) by the stepwise one-electron reduction of oxygen, showing the superoxide anion (O₂⁻), hydrogen peroxide (H₂O₂) and the hydroxyl (OH) radical. Of the shown intermediates, the hydroxyl radical is the most reactive and therefore damaging ROS species with a half-life of ≈1 nanosecond. It is formed in the so-called Fenton reaction in the presence of transition metals such as e.g. iron.

FIG. 20 Overview of some harmful effects of the hydroxyl (OH) radical, with a selection out of >100 pathologies in which ROS have been implicated.

### DEFINITIONS

As used herein, the term “ATP” is meant to include also related compounds or substances that are functionally equivalent with ATP, i.e. with a substantially similar profile of effect in processes as herein described, as well as pharmacologically acceptable salts thereof, or chelates thereof, or metal cation complexes thereof, or liposomes thereof, or incorporated in particles, e.g. for specific purposes such as drug targeting, in magnetic particles, incorporated in polymers such as DNA or RNA, etc. Examples of such related compounds or substances include analogues, derivatives and metabolites of ATP (including natural and synthetic compounds) that are functionally equivalent, for example purine and pyrimidine nucleotides such as UTP, GTP, CTP. Also included is a functionally equivalent combination of adenosine, AMP, and ADP, respectively, with phosphate, preferably inorganic phosphate. For particulars of such a combination, reference is made to refs. 9 and 10 the contents of which are herewith incorporated by reference.

### DETAILED DESCRIPTION OF THE INVENTION

The present invention is predominantly based on the observation that ATP exerts beneficial effects on the formation and scavenging of the extremely reactive and toxic hydroxyl (OH) radicals. Not only does our invention demonstrate that ATP favourably affects the formation and scavenging of OH-radicals in the Fenton type chemistry, it also attenuates OH formation from hydrogen peroxide which is formed in phagocytic cell cultures. In addition to these beneficial effects of ATP, we describe the empirical observation that, under different circumstances including conditions of severe oxidative stress, ATP inhibits the expression of pro-inflammatory cytokines such as TNF-alpha and stimulates the expression of anti-inflammatory cytokines such as IL-10.

It is now generally accepted that many chronic diseases and conditions in mammals and humans are associated with unbalanced production of reactive oxygen species (ROS), many (but not all) of which are free radicals. Radicals are produced under normal aerobic metabolism, mainly by leukocytes and by the respiratory chain in mitochondria, as well as from generation of NO by endothelium. In healthy humans, radicals are constantly produced, but this process is well regulated by scavenging abundant radicals via the antioxidant defense system. However, in conditions of metabolic stress, infections, disease or other aberrant conditions, an increased and unbalanced production of ROS often occurs. This phenomenon is called oxidative stress. Increased production of ROS during acute and chronic inflammation can further increase the oxidative stress.

The deleterious effects of ROS have been extensively reviewed (see (9), hereby incorporated by reference). ROS can produce acute damage to proteins, lipids and DNA. Oxidative stress renders proteins more susceptible to proteolytic degradation. ROS-induced lipid peroxidation in biomembranes can lead to changes in receptors and a cascade of intracellular events resulting in liberation in cytoplasm of nuclear transcription factor kappa B (NFκB), which controls gene transcription of acute phase mediators such as TNF-α. Oxidative stress also leads to oxidation of SH-moieties, not only in reduced glutathione (GSH) but also in membrane-bound Ca²⁺-ATPases (which provide an ATP-dependent active pumping system). Upon exposure to oxidative stress, the intracellular concentration of ATP decreases (10). Periods of oxidative stress are often followed by an increase in Ca²⁺-influx and intracellular Ca²⁺ levels, which can result in cell death. However, not only is increased ROS formation a trigger to cell death and inflammation, but inflammation itself again triggers radical production by different pathways. In this way, a vicious spiral of increased ROS formation, tissue damage, exhaustion of antioxidant reserves, and inflammation may occur.

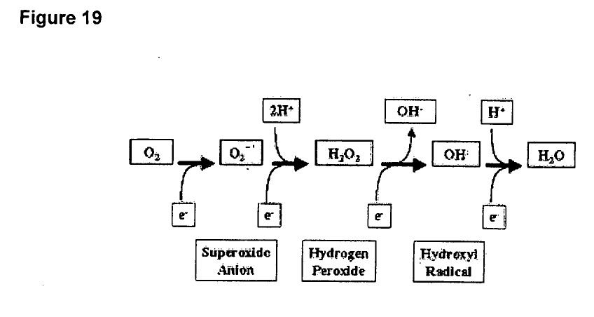

Different forms of ROS exist which are related (see FIG. 19, from (11)). A crucial trigger in the vicious spiral of ROS-induced damage are metallic ions, which are released during cell destruction, and which induce and amplify oxidative stress by converting hydrogen peroxide to the highly aggressive hydroxyl (OH) radical. Superoxide (O2-) radicals either dismutate to H₂O₂, and may thus lead to production of the hydroxyl radical, or may react with NO, which also yields the hydroxyl radical. The hydroxyl radical is known to be one of the most reactive forms of the reduced oxygen species (12). It is estimated that this radical has a half-life of ≈1 nanosecond in a biological environment. This implies that the hydroxyl radical will react with any bio-molecule in its environment, especially fatty acids, proteins, and nucleic acids such as DNA. Moreover, hydroxyl radicals can initiate the oxidative breakdown of poly-unsaturated fatty acids, leadings to the chain reaction of lipid peroxidation. This notion also explains the extreme toxic properties of the hydroxyl radical. Recent reports ascribe a major role for the metal ions such as iron in the aetiology of conditions such as Alzheimer's disease, cardiovascular disease and others.

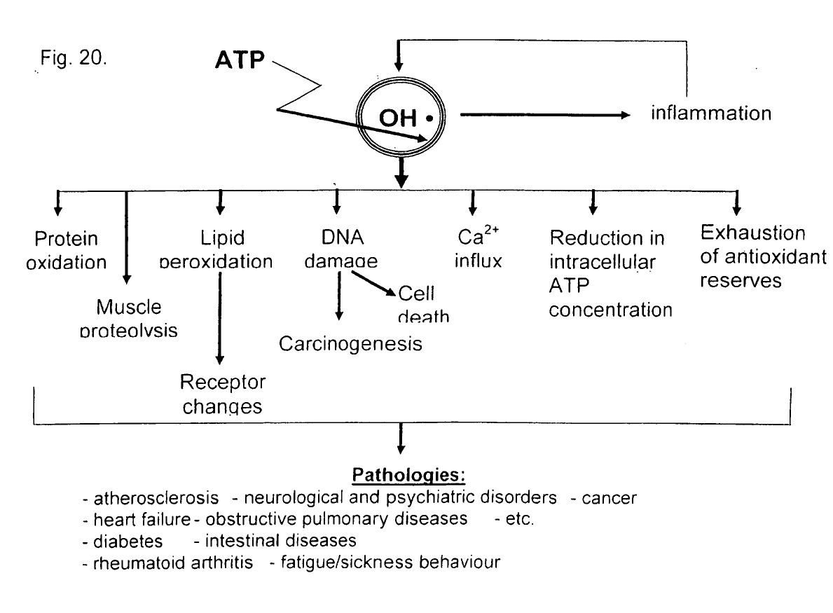

FIG. 20 illustrates the potential consequences of increased production of the hydroxyl radical. Increased and unbalanced ROS production has been implicated in the aetiology and progression of over a hundred pathological conditions; already more than a decade ago, Bast (9) mentioned disorders of the lung, brain, kidney, cardiovascular system, gastrointestinal system, liver, blood, eye, muscle, skin; and others, and many other diseases and conditions since then have been added to this list. Among a multitude of examples, we here mention just some conditions in which oxidative stress is known to be an important trigger of tissue damage, or a marker of disease progression and prognosis (13-28): obstructive lung diseases such as asthma and chronic obstructive pulmonary disease (COPD), conditions associated with intestinal dysfunction such as drug-induced intestinal damage, irritable bowel syndrome and inflammatory bowel disease (IBD); rheumatoid arthritis (RA) and osteoarthritis; radiotherapy/chemotherapy in cancer; the peri-operative inflammatory response; trauma; sepsis; the systemic inflammatory response syndrome (SIRS); ischaemia-reperfusion in different organs including the heart, intestine and brain; acute and chronic cardiovascular and cerbrovascular diseases; athero-sclerosis; heart failure; diabetes; syndrome X; obesity; aging; renal disease and chronic renal failure; anorexia; wasting conditions such as cachexia, kwashiorkor and sarcopenia with loss of lean body mass, muscle mass, muscle strength and/or fat mass; osteoporosis, fibromyalgia, autoimmune disorders, immune depression by e.g. surgery, HIV or AIDS; immune dysregulation, infectious and atopic diseases; chronic fatigue syndrome; neurological diseases such as Alzheimer's disease and Parkinson's disease; infectious diseases such as tuberculosis; sickness behaviour; and other inflammatory and pain syndromes. Increased ROS formation also plays a role subsequent to treatment with drugs; for example, increased intestinal permeability, a frequent side effect of oral non-steroid anti-inflammatory drugs (NSAIDs) is now considered to be associated with elevated ROS production. Many of the mentioned conditions constitute an great burden to individuals and the health care system, and lack of efficacy; side effects; and high cost (e.g. TNF-blockers) indicate the need for complementary treatment modalities which are effective, cheap and without side effects.

As a further example to illustrate the far-reaching consequences of oxidative stress, the effects of oxidative stress have recently been illustrated for COPD (28), based on evidence linking the wasting that occurs in COPD patients to both oxidative stress and oxidative stress-mediated processes, such as apoptosis, inflammation, disruption of the excitation-contraction coupling and atrophy.

Insight into the role of ATP in oxidative stress, immunity and inflammation in humans in vivo is very limited; existing knowledge derives mainly from in vitro studies. By stimulating purinergic receptors, ATP exerts various effects on different cell types. These studies have in general been performed using cultured cells or cell lines derived from humans or animals, and are directed towards unravelling biochemical mechanisms at molecular receptor and post-receptor levels, rather than providing a realistic picture of normal physiological or pathological situations in human subjects in vivo. Generally speaking, such studies in cultured cell lines and isolated cells are far away from the in vivo situation for a number of reasons. One such reason is that, due to repeated cell divisions, cell lines develop features which are distinct from in vivo human cells, for instance with respect to receptor expression and activity, intracellular cascades, transcription factors, etc. Furthermore, cell-to-cell interactions between different cell types, which play an essential role in determining physiological effects in the in vivo situation, are absent in such cell models.

Based on these studies, the direct effects of ATP according to the state of the art can be clearly summarized as oxidative stress-enhancing and pro-inflammatory. For instance, it is well established that ATP induces NO production by natural killer (NK) cells and macrophages. It is also well known that ATP promotes leukocyte phagocytosis by enhancing degranulation and stimulates the oxidative burst, i.e. the release of reactive oxygen and nitrogen species such as superoxide and H₂O₂ by different immune cells such as neutrophils, natural killer cells and macrophages (29-32), thereby not only inducing cell death in bacteria but also in normal cells.

The present invention is surprising and in contrast with the above prior art—which suggested that ATP promotes oxidative stress—in that we have now found for the first time that ATP favourably affects hydroxyl radical formation and scavenging from H₂O₂ during Fenton chemistry; even at concentrations as low as 100 μM, ATP induced a significant inhibition of the formation of the spin adduct DMPO—OH in electron spin resonance (ESR) experiments. Moreover, a concentration-dependent decrease in DMPO—OH spin adduct formation was observed by incubating with ATP, with a ≈80% inhibition at the highest ATP concentration used. Furthermore, in extensive testing, we found that the observed effect on DMPO—OH spin adduct formation decreased in the order ATP>ADP>AMP; also, we found that adenosine and adenine showed little or no effect on hydroxyl radicals.

As mentioned above, ATP is known to enhance the oxidative burst of neutrophils and other phagocytic cells. Previously we and others have found that ATP elevates free intracellular Ca²⁺ which can explain the stimulating effect on the oxidative burst (33, 34). In a study using guinea pig alveolar macrophages (according to (35, 36)) we confirmed the ATP dependent increase in H₂O₂ formation. We then incubated guinea pig alveolar macrophages with LPS in the presence of the spin-trap DMPO and found that ATP inhibited the DMPO—OH adduct formation in a concentration-dependent manner. This finding underlines the importance and practical relevance of the present invention that ATP attenuates of hydroxyl radical DMPO adduct formation in a more chemically oriented set-up. Our finding that the formation of the DMPO—OH adducts is inhibited by the presence of ATP, also in incubations with alveolar macrophages, is of great promise for the protective effect of ATP in diseases and conditions in which oxidative stress plays a predominant role, as discussed on the previous pages.

It is well-known that oxidative stress is an important trigger of an inflammatory response, through different mechanisms including liberation of NFκB, a process leading to gene transcription and release of pro-inflammatory cytokines. One of the most important pro-inflammatory cytokines is TNF-α. In contrast, interleukin-10 (IL-10) is considered as an important anti-inflammatory cytokine, the release of which therefore indicates inhibition of inflammation. For this reason, we tested the effect of ATP on the release of these two cytokines in whole blood ex vivo, a model closely resembling the in vivo situation. Since previous reports in patients with inflammatory disorders had demonstrated that anti-oxidant supplementation in these patients is able to inhibit the inflammatory response, we hypothesized that ATP, by virtue of its above favourable effect on OH formation and scavenging, would also inhibit inflammation, even under circumstances of severe oxidative stress. For this purpose, we used whole blood as a model which comes close to the in vivo situation, in contrast to previous studies in isolated blood cells or cell lines which are far away from the in vivo situation.

Results showed that ATP induced a dose-dependent inhibition of the inflammatory response to an insult inducing severe oxidative stress, such as H₂O₂ (5 mM) or γ-irradiation (16 Gy). In this patent application, severe oxidative stress is defined as H₂O₂ concentrations of about >1 mM, or radiation doses of about >10 Gy. In both circumstances of induced severe oxidative stress, ATP induced a reduction in the release of the pro-inflammatory cytokine TNF-α, relative to the anti-inflammatory cytokine IL-10.

In addition, we found in accordance with the present invention that ATP inhibits excessive inflammation by inhibiting the inflammatory response to an external insult such as LPS and PHA under circumstances of severe oxidative stress. To that end, in the same model as mentioned above, i.e. whole blood was incubated ex vivo with LPS and PHA in the presence of ATP and hydrogen peroxide (H₂O₂). ATP inhibited the LPS+PHA induced release of the pro-inflammatory cytokine TNF-α, and simultaneously induced a significant increase in the release of the anti-inflammatory cytokine IL-10 under these conditions. The results show for the first time that ATP inhibits the inflammatory response to a strong inflammatory insult such as LPS and PHA in the presence of severe oxidative stress, by modulating the cytokine production in whole blood. The observed response was highly consistent in different subjects. The same effect was found when blood was stimulated with LPS+PHA but without H₂O₂ or γ-irradiation. Similar to the marked beneficial effects of ATP in ESR experiments, the effects on cytokine production in blood decreased in the order ATP>ADP>AMP>adenosine.

Furthermore, we were able to reproduce the effects of ATP, as observed in whole blood from healthy subjects, by testing the effect of ATP in blood from patients with different oxidative stress-related diseases. Again, our results confirmed that in blood from these patients, ATP induced a reduction in the release of the pro-inflammatory cytokine TNF-α, relative to the anti-inflammatory cytokine IL-10. This result was found regardless of whether the blood from these patients was stimulated with LPS+PHA or not.

Moreover, we found that ATP reduces the intestinal permeability induced by NSAIDs in the small intestine of human subjects, as assessed by the lactulose/rhamnose (UR) intestinal permeability test. The effect of ATP is believed to be stronger than that of adenosine.

In addition it was found, as described in detail in the Experimental section, that ATP exerted certain new and surprising favourable clinical effects in patients with different conditions related to oxidative stress, including joint diseases such as rheumatoid arthritis; fatigue and exhaustion, including the full spectrum from chronic fatigue to pre-terminal cancer; and mood disturbances. Such favourable effects were also observed in pre-terminal cancer patients as well as in cancer patients undergoing cytotoxic and ROS-inducing treatments such as radiotherapy. In these conditions, ATP induced remarkable improvement with regard to a wide variety of symptoms; as a selection of such symptoms, we here mention improvement with respect to symptoms such as joint swelling, tenderness and stiffness; pain; self reliance including the ability to wash, dress, get in/our of a chair, or walk stairs independently, perform household activities, go for a walk, or go to work; normal intestinal function; dry/sore mouth; and mental state mood, ability to concentrate and to memorize normal daily issues, neurological functioning, worrying, dizziness, decreased sexual interest, tension, and sleeping difficulties.

Furthermore, we surprisingly found that, in patients with advanced cancer, ATP induced normalization of several other blood parameters including lactate dehydrogenase (LDH). LDH is considered as an indicator of tumour progression and a prognostic marker of survival in several types of cancer e.g. (37, 38). This effect of ATP may enhance the previously described favourable clinical effects of ATP. ATP also corrected hypertriglyceridaemia in these patients, which is not only a well-known part of the paraneoplastic syndrome but also part of the insulin resistance syndrome (syndrome X) and diabetes, another condition which is closely linked to oxidative stress.

Furthermore, we found that ATP attenuates the irradiation-induced decrease in GSH and GSH/GSSG ratios, as well as attenuation of radiation-induced pro-inflammatory reaction in whole blood, as shown by inhibition of TNFα stimulation in irradiated blood, relative to control blood samples which were irradiated but not treated with ATP.

Thus, in preventing and treating certain clinical conditions and diseases, ATP and related compounds can inter alia be used in the framework of the present invention in any condition that is or will be associated with oxidative stress in any part of the body, such as have been mentioned in this section above.

Although the inventors do not wish to be bound to any theory, it is believed based on their experiments that the above effects of ATP are caused by a concerted inhibition of two major processes by ATP:

1. First and predominantly, ATP prevents or attenuates oxidative stress, based the observation that ATP has a beneficial effect on the formation and scavenging of the extremely reactive and damaging hydroxyl (OH) radicals. Thus, by different mechanisms including preventing the conversion of H₂O₂ to the highly aggressive hydroxyl radical, ATP prevents the induction and amplification of oxidative stress as induced by different pathways, such as by metallic ions released during cell destruction, and by the mitochondrial chain. As a consequence of the interference with increased ROS formation by ATP, not only will ATP prevent cell damage, but also moderate the excessive inflammatory response to these processes.

2. The above effect of ATP is further supported by an additional virtue of ATP, i.e. direct inhibition of the inflammatory response due to specific stimulation of P2 purinergic receptors by ATP, possibly in combination with indirect effects through P1 purinergic receptors. In addition, ectoenzymes such as ecto-ATPase may act as signaling molecules which, upon stimulation by ATP, inter alia regulate effector functions of immune cells such as lymphocytes. Mechanisms of ATP-induced favourable effects may inter alia include regulation of membrane pore formation; cyclic AMP- and/or calcium²⁺ mediated pathways; signal transduction through inositol phosphate and related compounds; transcription pathways related to nuclear factor kappa B (NFκB); inhibition of poly(ADP-ribose) polymerase (PARP), mitochondrial pathways; and the like.

In conclusion, ATP is more than a simple antioxidant: it interferes with radical formation and thereby exerts beneficial effects in controlling oxidative stress as well as the inflammatory response and immune competence within the mammalian body. Thus, in situations where excessive oxidative stress is accompanied by exhaustion of the immune system, ATP will induce immune activation by its beneficial effects on OH formation and scavenging. In contrast, in situations where acute or strong oxidative stress induces an excessive or aberrant inflammatory response, such as after trauma or surgery, in inflammatory and pain conditions, in rheumatoid arthritis, in autoimmune disorders, atopic disease, etc. etc., or any other conditions such as discussed on the previous pages, extracellular ATP helps in dampening, normalizing or terminating the inflammatory process by virtue of its effect on OH radical formation and scavenging.

A practical application of these findings which form part of the present invention is the use of, for example, varying ATP infusion rates, at different duration, frequency, dosage, dosing-time schedule route of administration, etc. in order to achieve differential effects in different immune-related conditions. Especially, it is noted that doses of ATP of ≦40 μg/kg·min show surprising efficacy.

### New Uses of ATP

The findings indicate that, in addition to the previously described anabolic properties of ATP, ATP is potentially useful as an oxidative stress-preventing, tissue-protecting and immuno-modulating drug under varying conditions of oxidative stress, including inter alia in the prevention and treatment of the following conditions, part of which have been previously mentioned: intestinal damage and similar conditions associated with oxidative stress, including amongst other things the damage induced by NSAIDs or other insults, medications or substances (e.g. alcohol, exercise, smoking, etc.) in healthy and diseased subjects, diarrhoea, obstipation, irritable bowel syndrome, and different forms of inflammatory bowel disease such as, but not restricted to, Crohn's disease and ulcerative colitis; rheumatoid arthritis and similar conditions (as outlined above); obstructive pulmonary diseases and similar conditions (as outlined above). We describe herein long-term favourable effects of low-dose ATP infusion in contrast to the state of the art, which only relates to immediate bronchoconstrictive effects induced by inhalation of adenosine or ATP; cancer, where ATP treatment in combination with (i.e. before, during or after) radiotherapy, chemotherapy or surgery, will reduce the oxidative stress and inflammation as caused by these treatments in apparently healthy host tissues, leading to, amongst other effects, a reduction in short and long term physical side effects such as dry/sore mouth, obstipation, and fatigue; an increased appetite; an improved nutritional status, improved tumour control (tumour response/time to progression) and prolonged survival; conditions related to the neurological and mental state and functioning which are related to oxidative stress and/or ROS production, such as: fatigue, burn out; to improve sleep quality and prevent or treat sleeping difficulties; to enhance concentration or resolve problems of concentration; to prevent and treat dementia, depression, and/or anxiety; to prevent and treat other mood-related conditions such as inter alia worrying, despair, irritability, tension, stress; disorders related to balance such as dizziness; fibromyalgia; muscle tenderness; energy; decreased sexual interest, or similar conditions; sickness behaviour; conditions related to temperature regulation such as shivering; conditions related to mountain sickness and hypobaric hypoxia such as may be present at high altitude; conditions occurring in aircraft, space shuttles, and the like; atopic diseases and allergies; peri-operative inflammatory response, trauma, endotoxaemia in healthy and diseased subjects, systemic inflammatory distress syndrome (SIRS), acute and chronic cardiovascular diseases, atherosclerosis, heart failure, syndrome X, aging, endocrine pancreatic disorders, obesity, anorexia; wasting conditions such as cachexia and sarcopenia; osteoporosis, fibromyalgia, infectious diseases, other inflammatory and pain syndromes, auto-immune disorders, skin disorders, immunosuppression due to surgery, HIV infection, AIDS, and other similar conditions; in the treatment of unwanted side effects of anti-inflammatory and immunosuppressive drugs, for instance in when treating the immuno-incompetence or limited resistance to infections as a consequence of administration of these drugs; mountain sickness and related syndromes; in air planes (air sickness), boats (sea sickness), and space shuttles.

In general, it is expected that these effects of ATP will not only aid in the primary, secondary and tertiary prevention and treatment of diseases and disorders, thus reducing the burden and suffering of patients, but also contribute to lowering health care costs and increasing work participation in some of the aforementioned chronic inflammatory diseases and conditions as well as other immunological disorders, burn out syndrome, etc.

### Preparation and Administration of ATP and Compositions Comprising ATP

When applying ATP in accordance with the present invention in mammals, preferably human beings, the medicine is usually and conveniently in the form of a pharmaceutical or nutritional composition, preferably a pharmaceutical composition for oral or parenteral administration. The pharmaceutical composition for parenteral administration is preferably adapted for continuous infusion of ATP, more preferably in an amount up to 150 μg/kg·min for regular administration, the composition further comprising a pharmaceutically acceptable carrier. The amount of ATP in nutritional compositions (or food supplements) is preferably subdivided in dosages of up to 25 g/day for regular administration.

Pharmaceutical and nutritional compositions comprising ATP can be prepared by any convenient manner which is known to a person skilled in the art. In one preferred embodiment of the invention, a pharmaceutical composition is formulated as the disodium salt of ATP (ATP-Na₂). In another preferred embodiment, a pharmaceutical composition is formulated as a lyophylized preparation of ATP-Na₂.

We have now developed and tested ways and methods to safely administer ATP by intravenous infusion in the setting of private homes, i.e. without direct medical supervision, by a trained nurse. Within the framework of the present invention a training programme for nurses is provided to safely prepare and administer ATP solutions by intravenous infusion. In a preferred embodiment of the invention, after one ATP infusion course which is preferably administered under medical supervision, subsequent ATP infusions can be given without medical supervision e.g. in the home setting by a trained nurse. The said training programme for nurses has been developed to safely prepare and administer ATP solutions by intravenous infusion. This programme has been tested in home care organizations in four different regions within the European Union, demonstrating that this is not dependent on the region or country provided trained nurses supported by a hospital, nursing home, home care organizations or any comparable professional health care organization exists.

In one preferred aspect of the invention, ATP is administered in combination with phosphate in either inorganic, organic or any other form during the same period of time, in subsequent order, or alternating. In particular, Rapaport (4) has described that adenosine administered in combination with phosphate inhibited host weight loss of tumour-bearing animals to a similar extent as ATP, whereas adenosine without phosphate was ineffective. Based on this prior art, we expect that administration of nucleosides such as adenosine in combination with inorganic phosphate will also be similarly effective as ATP.

Freeze-drying can be performed in any conventional way which is known to a person skilled in the art. In a preferred embodiment of the invention, freeze-drying is performed in a KLEE freeze dryer essentially according to the following procedure:
1. Sterilized standard freeze-drying stoppers are pre-treated for 24 hours at 110° C. to remove moisture;
2. Solutions of mannitol in the range of 0.01% to about 25%, preferably 1.5 to 6%) and/or HES in the range of 0.01% to about 25%, preferably 1.5 to 3%, and/or sucrose, in the range of 0.1% to 25%, preferably 3 to 5%, and/or trehalose, in the range of 0.1% to 25%, preferably 3 to 5% are prepared with distilled water (other filler(s) known in the art can be used alternatively);
3. ATP is added to these solutions (preferably in concentrations from about 1 g to 8 g/10 ml);
4. Sterilized freeze-drying vials are filled with an amount of a solution containing 1 to 8 g ATP, using a calibrated Gilson pipet or other adequate equipment;
5. Vials are stoppered with standard rubber stoppers;
6. Vials are stored at ambient temperature for up to 1 hour;
7. Vials are placed on shelves of the freeze-dryer which are precooled to −38° C.;
8. Freezing of the solutions is performed for 45 min on the precooled shelves;
9. The freeze-drying cycle is then started;
10. After lowering the chamber pressure in the freeze-dryer to 8×10⁻² mbar, the temperature is kept at −18° C. during primary drying phase;
11. During the secondary drying phase, the process is controlled using pressure raise testing.

In contrast to crystalline ATP and ATP in solution, the lyophilized ATP preparation is stable at room temperature for at least 1 to 3 years. It can be easily dissolved in saline, and thus the infusion solution can be prepared fresh by a trained nurse even in a non-clinical setting. In this way, it is logistically feasible and safe to administer ATP in the setting of a private home, nursing home, etc. by a trained nurse, without need for medical intervention.

In another preferred embodiment of the invention, ATP is administered as a series of about 1 to 20 intravenous infusions at intervals of about 1 to 4 weeks.

In order to determine the tolerance for ATP as well as the maximally tolerated dose of ATP, the first ATP infusion is preferably administered under medical supervision, usually in an in- or outpatient setting. Subsequent infusions can either be started at the hospital day care centre, at private homes, nursing homes, etc. according to a standardized protocol. Our experience shows, for the first time, that it is feasible and safe to administer subsequent ATP infusions in the home setting. In a total of over 60 home infusions in cancer patients, no serious side effects grade 3-4 on the WHO Common Toxicity Criteria scale occurred. No hospital admissions were necessary.

The preparation may be given as an intravenous infusion of 0.01-150 μg of ATP etc. per kg body weight per minute, at a frequency varying from continuous infusion to low frequency (e.g. once per year). A suitable infusion time and frequency is, for example, 8-12 hours or 24-30 hours of ATP infusion once per week or once per 2-8 weeks. Another suitable frequency is, for example, 1 minute to 4 hours every day, every second day, or on several days per week, for a certain period, with or without days of interrupting the infusions. Instead of intravenous infusion, other routes of administration may be preferred: intraperitoneal, subcutaneous, oral, topical, nasal, sublingual, as a spray, as tablets, emulsions, and the like.

In a further preferred embodiment of the invention, intravenous infusion of ATP is initiated at an infusion rate ranging from about 5 to about 40 μg/kg·min, preferably of about 20 μg/kg·min which is subsequently increased by steps ranging from about 5 to about 20 μg/kg·min, preferably of about 10 μg/kg·min every 5-30 min., preferably about 10 min. If side effects appear, the infusion rate is reduced in steps preferably of about 10 μg/kg·min every 5-30 min (preferably about 10 min) to the dose where side effects have fully disappeared. This dose is the maximally tolerated dose, which essentially has to be determined individually in each subject.

According to the present invention, the frequency, duration and rate of ATP infusion may be varied in order to achieve desired specific effects. A suitable approach is to vary the dose, duration, frequency etc. within one patient according to his/her specific needs. For instance, in one preferred aspect of the invention, when aiming at increasing muscle strength, a dosage of about 75 μg/kg·min may be applied, whereas a dosage between about 40 to 60 μg/kg·min may be given when aiming at ameliorating shortness of breath, constipation, fatigue or quality of life, and a much lower dosage (e.g. 10-15 μg/kg·min) in the treatment of joint swelling and fatigue in patients with rheumatoid arthritis or chronic fatigue syndrome. Variations in dosage and/or concentration of ATP, further ingredients of the composition, frequency, etc. depend on several individual factors of the individual to be administered, such as age, sex, condition of the individual, and are usually determined on an individual basis by a physician or other skilled person. The ATP solution may contain ATP in the form of one or more salts, e.g. mono- or di-Na-ATP, Mg-ATP or the combination of ATP etc. with MgCl₂, preferably in conjunction with a pharmaceutically acceptable carrier or vehicle and/or other ingredients which are known to a person skilled in the art. Other ways of increasing intra- or extracellular ATP levels, for instance by stimulation of ATP production or release, may also be applied in accordance with the present invention.

In accordance with the present invention ATP and/or derivatives can be applied in parenteral and enteral nutrition, alone or in combination with specific compounds comprising those mentioned within this application. The preparation of such compositions is well known to people skilled in the art and can be optimized in a routine way without exerting inventive skill and without undue experimentation. The dosage and frequency of administration depends inter alia on well-known factors, such as the weight of the individual to be administered, age, sex, condition, etc., and will usually be determined by a physician or other person skilled in the art.

Other substances may be given simultaneously in the same pharmaceutical or nutritional preparation which comprises ATP. Another possibility is that various treatment schedules are developed in which administration of ATP and other components may be given during the same period of time, in subsequent order, or alternating, etc. Such other compounds include, for example, phosphate in either inorganic, organic or any other form; n-3 fatty acids such as eicosapentaenoic acid (EPA), docosahexaenoic acid (DHA) and/or alpha-linolenic acid, preferably administered as triacylglycerol, but also as free fatty acids or esters, for example ethyl esters, if desired; creatine; one or more amino acids, such as: cyst(e)ine, preferably as N-acetyl cysteine (NAC), but also other cyst(e)ine derivatives; arginine; glutamine; glutamate; and/or other amino acids; carbohydrates, such as ribose and others; antioxidant vitamins such as vitamin C, vitamine E and others; other antioxidants such as carotenoids, flavonoids, isoflavonoids, phyto-estrogens, and others; minerals and trace elements such as selenium, calcium, magnesium, and others; nutrients, non-nutrients, pharmacological compounds; and the like.

The ATP-containing pharmaceutical compositions which are useful for the purpose of the present invention may additionally comprise one or more substances selected from the group of stimulants, hormones, analogues of such hormones, phyto-hormones, analogues of such phyto-hormones, or other pharmacological compounds of choice, which are all within the realm of a person skilled in the art based on his knowledge, experience and/or experimenting without inventive effort.

### Infusion Device

The inventors also wish to proclaim another preferred embodiment of the invention, in which a special device is use adapted to specific needs of ATP administration in a non-clinical setting such as private homes. For this purpose, an infusion pump is developed which meets the following requirements: less than 100 g of weight; can be programmed in advance and on-the-spot to build up the infusion dose in steps of 5-20 μg/kg·min; allows data entry of patient weight, concentration of infusion solution, and ATP dose in μg/kg·min, and transfers these data to infusion rate (ml/hr); registers and saves the dose per minute over the complete infusion period, and calculates the minimal and maximal infusion dose, the infusion dose per hr, and the total administered ATP dose; can be programmed and handled at a distance using a wireless device; allows the patient to reduce the dose, but not to increase the dose. Also, an easy-to-wear bag is developed such that it allows wearing in a tailor-made fashion (waist, hip, back, etc.), fitting the infusion pump and an infusion bag (100-1000 ml).

### EXPERIMENTAL SECTION

To demonstrate the marked effects of ATP a model was used that simulates the in vivo situation, i.e. whole blood ex vivo.

### Experiment 1

### Methods

Electron spin resonance (ESR) studies were performed at room temperature using a Bruker EMX 1273 spectrometer equipped with an ER 4119HS high sensitivity cavity and 12 kW power supply. The following instrument conditions were used: scan range, 60 G; center magnetic field, 3490 G; modulation amplitude, 1.0 G; microwave frequency, 9.86 GHz; time constant, 40.96 ms, scan time, 20.48 ms; number of scans, 50. OH radicals were generated by the Fenton reaction, and 5,5-Dimethyl-1-pyrroline N-oxide (DMPO) was used for trapping hydroxyl radicals. Fifty microliters of 10 mM H₂O₂, 50 μl 250 mM DMPO, 50 μl milliQ, 50 μl milliQ (blanco) or sample and 50 μl 5 mM FeSO₄/5 mM EDTA were mixed, transferred to a capillary glass tube and the DMPO—OH spin adducts were measured after 2 minutes by ESR. Quantification of the spectra (in arbitrary units) was performed by peak integration using the WIN-EPR spectrum manipulation program.

### Results

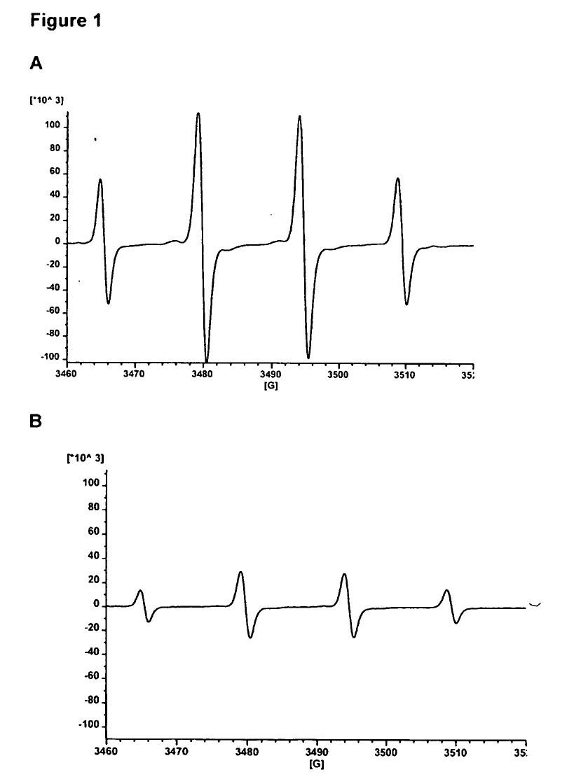

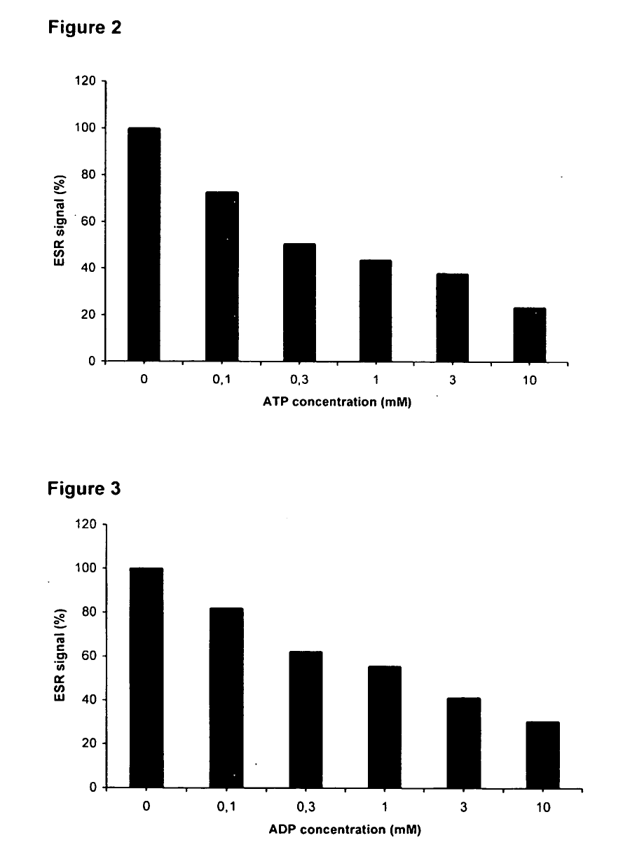

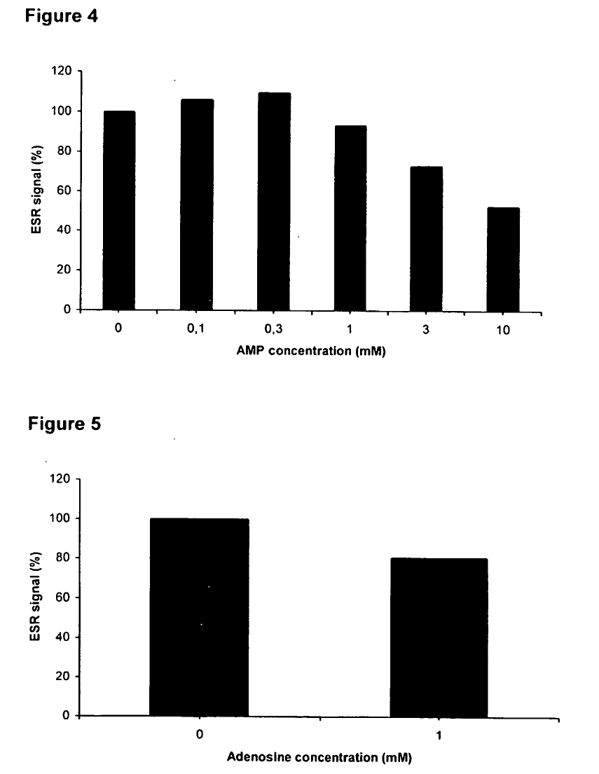

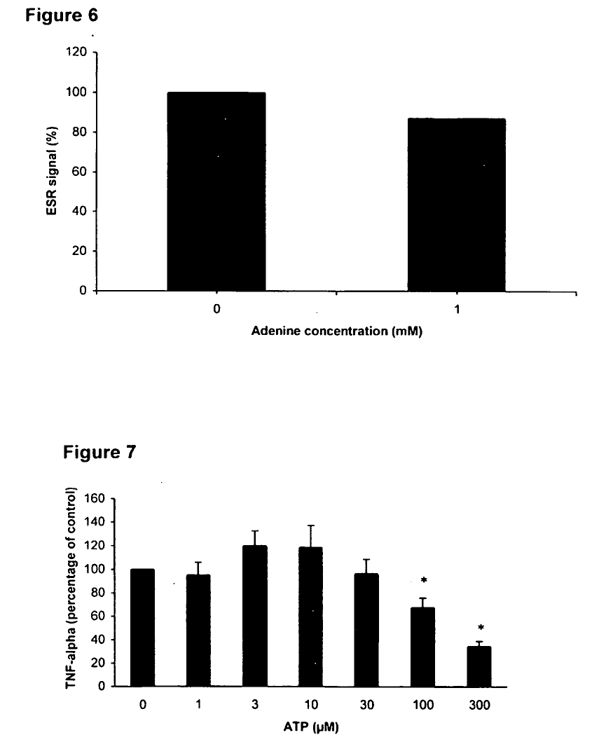

As shown in FIG. 1, ATP (panel B) strongly inhibited the DMPO—OH spin adduct formation generated by the Fenton reaction in the presence of iron, when compared with the control condition (buffer, panel A). As shown in FIG. 2, even at concentrations as low as 100 μM, ATP induced a significant inhibition of DMPO—OH spin adduct formation generated by the Fenton reaction. Moreover, a concentration-dependent decrease in DMPO—OH spin adduct formation was observed by incubating with ATP, with a 80% inhibition at the highest ATP concentration used. A concentration-dependent decrease in DMPO—OH spin adduct formation was also observed by incubating with ADP (FIG. 3), although ADP was slightly less effective than ADP in preventing DMPO—OH formation. AMP (FIG. 4) showed a reduction of hydroxyl radicals at 3 and 10 mM, but had no effect on hydroxyl radicals at lower concentrations (0.1, 0.3 and 1 mM). Adenosine (FIG. 5) and adenine (FIG. 6) showed little or no effect on hydroxyl radicals at the concentration of 1 mM.

### Experiment 2

### Methods

Blood was collected from 8 healthy subjects in heparin containing vacutainer tubes. All incubations were performed in duplicate. ATP was dissolved in RPMI 1640 culture medium, at a final concentration of 1-300 μM, and blood pre-incubated with ATP at 5% CO₂ at 37 DC for 30 min. After stimulation with LPS en PHA (10 and 1 μg/ml final concentration, respectively), the plates were incubated in 5% CO₂ at 37° C. for 24 h. Cell-free supernatant fluids were then collected by centrifugation (6000 rpm, 10 min at 4° C.) and stored at −20° C. until tested for presence of cytokines. All cytokines were quantified by means of PeliKine Compact human ELISA kits (CLB/Sanquin, The Netherlands), based on appropriate and validated sets of monoclonal antibodies.

### Results

Blood concentrations of TNF-α, IL-6 and IL-10 were low in control (i.e. not stimulated) samples and increased significantly under LPS+PHA stimulation.

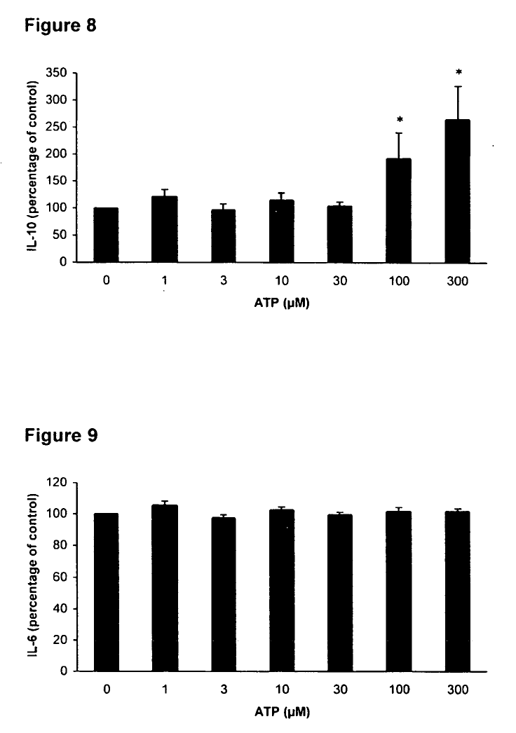

In blood pre-incubated with ATP, induced a dose-dependent inhibition of the release of the pro-inflammatory cytokine TNF-α in LPS-PHA stimulated whole blood at ATP concentrations of 100 and 300 μM (FIG. 7). At 300 μM ATP, a 65% inhibition of the TNF-α production was found. Moreover, ATP induced a dose-dependent rise in the release of the anti-inflammatory cytokine IL-10 in LPS/PHA stimulated whole blood at 100 and 300 μM ATP (FIG. 8); at 300 μM of ATP, we found a 62% stimulation of the IL-10 production. No effect of ATP on the production of IL-6 was found (FIG. 9).

### Experiment 3

### Methods

Whole blood of healthy subjects was collected as described for Experiment 2, and pre-incubated with 1 or 10 mM H₂O₂ at 5% CO₂ and 37° C. for 15 min. Then, ATP was added to the blood at final concentrations of 1-300 μM for the 30 min incubation step, and then incubated as in Experiment 2 with LPS/PHA for 24 hours.

### Results

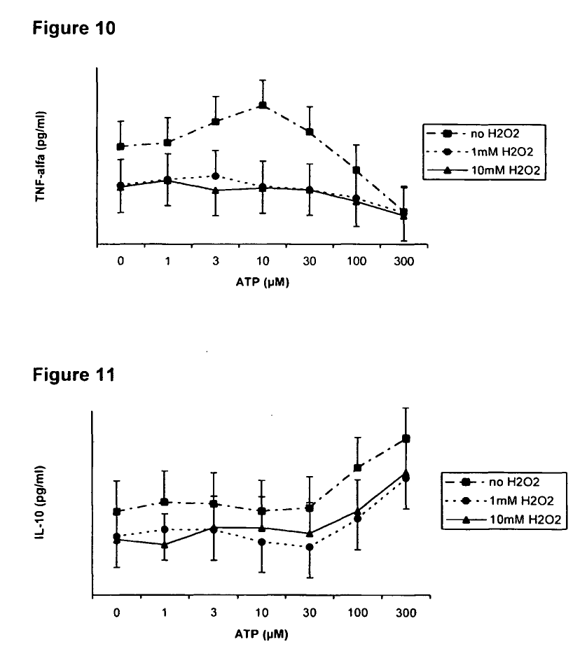

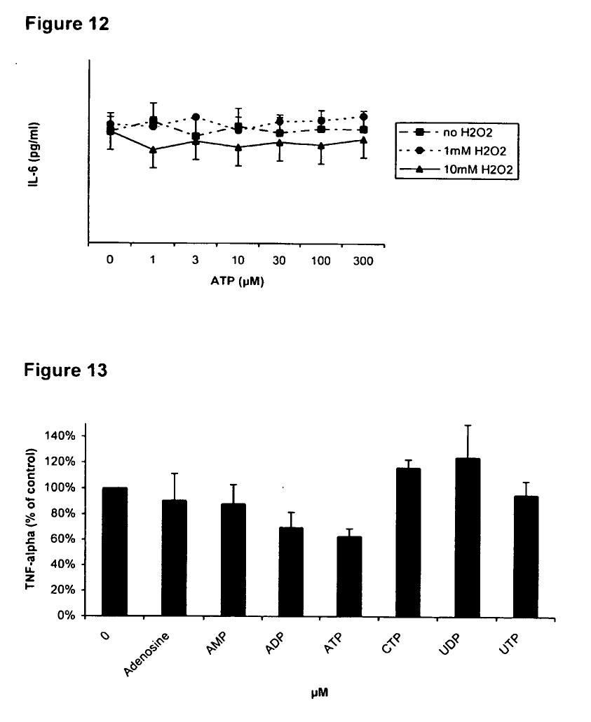

Incubation with LPS/PHA in the presence of 1 or 10 mM H₂O₂ without ATP induced a strong release of TNF-α, IL-6 and IL-10. In the presence of 1 or 10 mM H₂O₂, ATP significantly inhibited TNF-α release from LPS-PHA stimulated whole blood in a dose-dependent fashion (FIG. 10), with ≈50% inhibition of TNF-α release at 300 μM ATP. In the presence of 1 or 10 mM H₂O₂, ATP also induced a significant dose-dependent increase in IL-10 release (FIG. 11), with 50-60% increase at 300 μM ATP. ATP concentrations below 100 μM in combination with 1 or 10 mM H₂O₂ did not affect TNF-α release. ATP had no effect on IL-6 release from LPS-PHA stimulated whole blood either in the absence or presence of H₂O₂ (FIG. 12).

### Experiment 4

### Methods

Whole blood was collected and stimulated with LPS+PHA as described for Experiment 2. ATP, dissolved in RPMI 1640 culture medium, was added to the blood at a final concentration of 1-1000 μM. The agonists were added in the same way as ATP, however their stock solutions are prepared in PBS and further diluted in medium. All incubations are performed in duplicate.

### Results

Pretreatment of whole blood with ATP was more effective in inhibiting TNFα and stimulating IL-10 production in LPS-PHA stimulated whole blood, than ADP, AMP or adenosine (FIG. 13). CTP, UDP and UTP had little or no effect. The results of this experiment indicate that the effects of ATP are stronger than those of ADP, AMP and adenosine.

### Experiment 5

### Methods

This experiment was performed to test the effects of ATP under circumstances of oxidative stress in healthy subjects, but without LPS-PHA induced stimulation of cytokine production. Whole blood was collected as described for experiment 2, and was pre-incubated with ATP at final concentrations of 100-300 μM, or no ATP (control), for 30 min. Then, blood was incubated with H₂O₂ (5 mM) or 24 h, without addition of LPS+PHA. and incubated for 24 hours with 5 mM H₂O₂ at 5% CO₂ and 37° C. Cytokine production was measured as in Experiment 2. Results were expressed as the TNF-α/IL-10 ratio relative to the control condition (H₂O₂ without ATP).

### Results

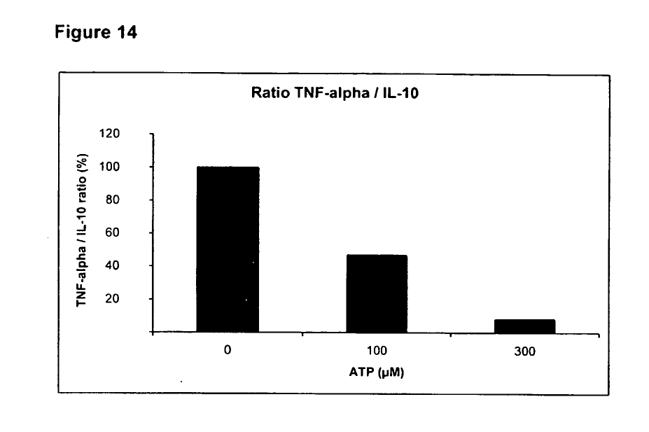

H₂O₂, added to whole blood, in the absence of both ATP and LPS+PHA, induced a slight increase in the TNF-α/IL-10 ratio, suggesting stimulation of inflammation by H₂O₂-induced oxidative stress. In the presence of H₂O₂ (with no LPA+PHA added), ATP induced a dose-dependent reduction in the TNF-α/IL-10 ratio (FIG. 14), with a 90% reduction in the TNF-α/IL-10 ratio at 300 μM ATP.

### Experiment 6

### Methods

This experiment was performed to test the effects of ATP ex vivo in whole blood collected from patients with oxidative stress-related diseases, both without and with LPS+PHA stimulation of cytokine production. Whole blood from 3 patients with different oxidative stress-related diseases was collected as described for experiment 2, and incubated for 24 hours at 5% CO₂ and 37° C., with ATP added at a final concentration 300 μM, or no ATP (control). Cytokine production was measured as in Experiment 2. Results were expressed as the TNF-α/IL-10 ratio relative to the control condition (no ATP).

### Results

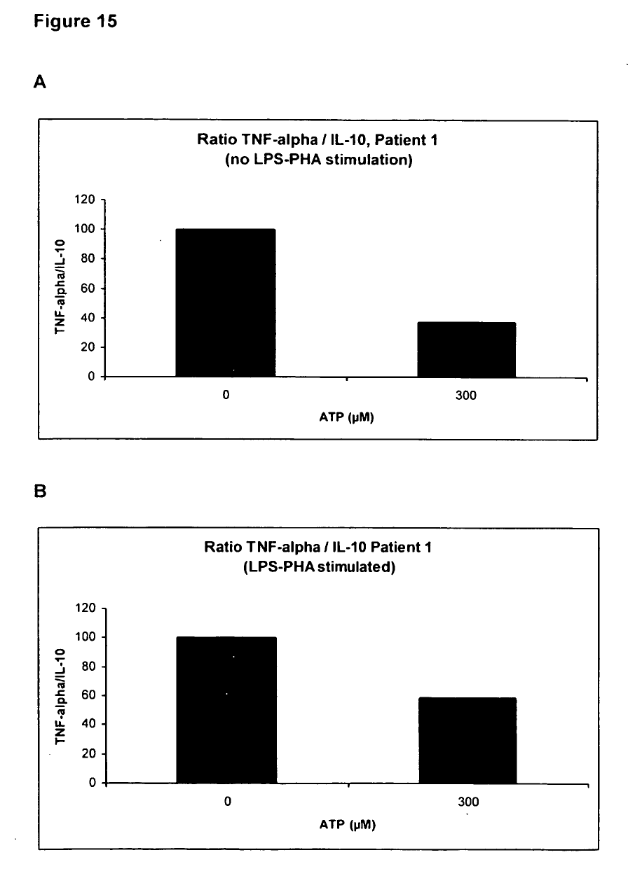

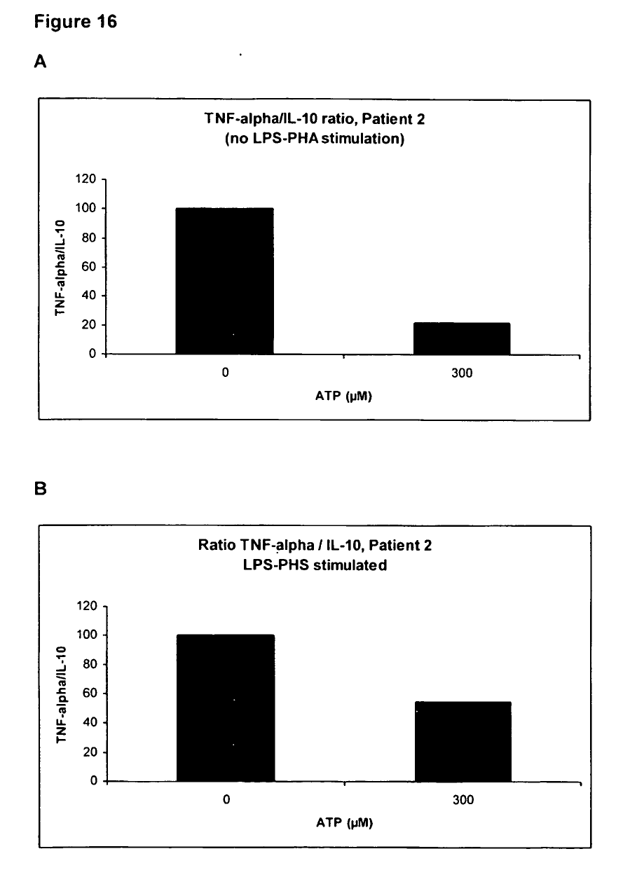

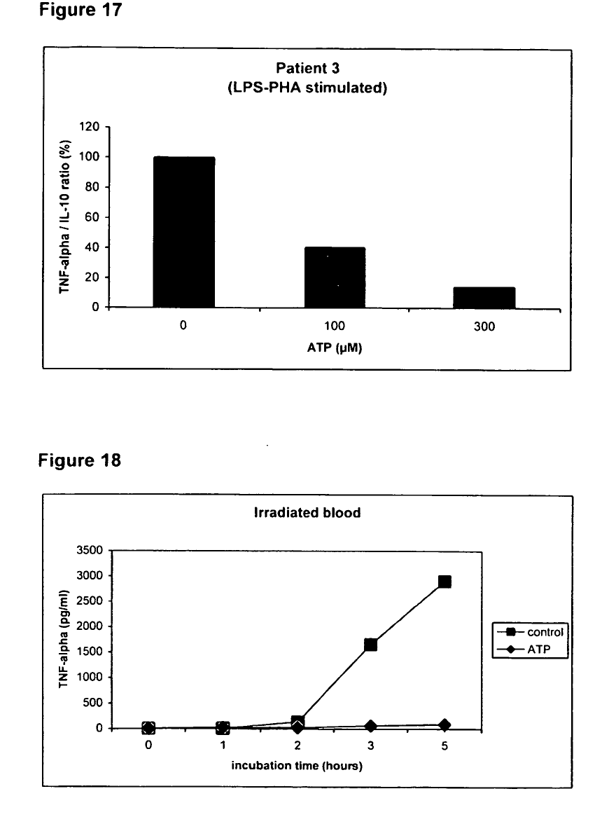

In blood incubated without LPS+PHA, ATP at a concentration of 300 μM induced a 60-80% reduction in the TNF-α/IL-10 ratio (FIGS. 15A, 16A). In blood stimulated with LPS+PHA, ATP induced a 40-80% reduction in the TNF-α/IL-10 ratio depending on the patient (FIGS. 15B, 16B, 17).

### Experiment 7

### Methods

Samples with 5 ml of blood were pre-incubated with 300 μM ATP or medium (control) for 30 minutes. Then, at t=0, each blood sample was irradiated with γ-radiation at a dose of 16 Gy. Preceding irradiation (baseline) and at 1 h, 3 h and 5 h post-irradiation, a sample was taken for analysis of intracellular GSH and GSH/GSSG ratios according to standard methods, as well as for cytokine analysis by the ELISA method.

### Results

In ATP-treated samples, relative to control samples (no ATP), attenuation of the irradiation-induced decrease in GSH and GSH/GSSG ratios was found. The marked irradiation-induced TNFα stimulation at 3 and 5 h post-irradiation was completely blocked by ATP (FIG. 18), demonstrating that ATP inhibits the inflammatory response of whole blood to γ-irradiation.

### Experiment 8

### Methods

Intestinal permeability is tested in healthy non-smoking human subjects using the lactulose/rhamnose (UR) intestinal permeability test. This barrier function test is based on the comparison of intestinal permeation of molecules of different sizes by measuring the ratio of urinary excretion of the disaccharide lactulose and the monosaccharide rhamnose. These two sugars follow different routes of intestinal permeation, i.e., lactulose permeates through the paracellular pathway, whereas rhamnose permeates transcellulary.

The experiments are performed as follows: at t=−14 hrs, a Bengmark-type naso-intestinal tube (Flocare, Zoetermeer, The Netherlands) is installed into the stomach. Next, at t=−10 hrs, subjects ingest a capsule of indomethacin (75 mg) immediately followed by administration of either ATP or placebo directly into the subject's duodenum through the inserted tube. At t=−1 hr, after an overnight fast, subjects receive a second dose of indomethacin (50 mg) followed by either ATP or placebo. Then, at t=0, the permeability test is performed as follows: subjects ingest a hyperosmolar drink containing 5 g of lactulose and 0.5 g of L-rhamnose dissolved in 100 ml water. After ingestion of the hyperosmolar test drink, total urine produced over 5 hours is collected.

### Results

It is expected that the urinary concentration ratio of lactulose relative to rhamnose in subjects treated with indomethacin and ATP will be lower than is the same ratio in subjects treated with indomethacin only.

### Clinical Cases

### Methods

Patients with different diseases received infusions of 10-75 μg/kg·min over 8-24 h, at intervals of 1-4 weeks in different randomized clinical trials. In all cases, the first. infusion was given under clinical supervision, subsequent infusions were given in the setting of private homes. Preliminary results in small numbers of patients with persistent rheumatoid arthritis, chronic fatigue syndrome, and cancer in the pre-terminal stage, and lung cancer during curative radiotherapy are given below.

### Results

### 1. Rheumatoid Arthritis

A female patient, 50 years and mother of 4 children, with seropositive, non-erosive RA with severe functional impairment of performance and exhaustion despite methotrexate (15 mg/wk) received a total of 10 ATP infusions at intervals of one week, dosage 10-15 μg/kg·min. After 10 infusions, the rheumatologist reported a spectacular improvement: joint swelling and pain had markedly decreased, physical examination showed minimal swelling of only a few joints, without tenderness at pressure; complaints of pain, stiffness and fatigue had almost disappeared and the patient was able to function normally. DAS score had decreased from 5.80 to 3.09 and CRP had decreased from 43 to 6 mg/L.; all other blood values were normal. MD's conclusion: marked decease of disease activity.

The subjective report by the patient regarding activities in daily living and quality of life included the following: before ATP-treatment, the patient felt extremely tired, mentally diffuse, and had difficulties in concentrating and memorizing normal daily issues; was unable to take a shower, undress or dress independently, to walk up stairs, to stand up from a chair, or to perform light household activities such as cleaning windows, vacuum cleaning, or lifting a pan. After 8 ATP infusions, the patient reported to be able to perform all of the mentioned activities independently, and besides to go for shopping; to go for a beach walk, to be able to concentrate and perform the financial administration, as she had not done for many years.

### 2. Chronic Fatigue Syndrome (ME)

In a double-blind design, 8 out of 9 patients with chronic fatigue syndrome who had been treated with ATP (8 infusions of 24 h), spontaneously reported the following unexpected beneficial effects of ATP infusion:
less pain on the day after the infusion
feeling better during the 8-week infusion period than before or afterwards
more physical energy on the day after the infusion
felt better during the complete infusion period: less pain, better performance status, happier, mentally stronger, less tired, sleeping better
fewer ulcers during the infusion period
feeling less weak/feeble on the day after the infusion, feeling muscles “in a positive sense”
   In 5 out of 9 patients, these effects were already noted on the day after the first ATP infusion.
   These effects were noted at surprisingly low dosage of ATP, often 10-25 μg/kg·min. In the placebo group, not even one out of 9 patients spontaneously reported any of such improvements.

### 3. Pre-Terminal Cancer Patients

This study was performed in patients of different tumour types with a life expectation of between 8 and 12 weeks. Patients received a maximum of 8 ATP infusions (8 h) at an infusion rate of max. 50 μg/kg·min. Preliminary data analysis showed that 5 out of 19 patients who had completed >4 ATP infusions had spontaneously reported marked improvements, despite ATP doses which were in some patients lower than in previous studies. These subjective improvements were supported by objective outcome assessment using validated questionnaires for self-reliance (Groningen Activity Restriction Scale, GARS), fatigue (Short Fatigue Questionnaire, Smets et al.), and appetite (visual analogue scale, VAS). Below, some of the major effects in these five patients are summarized.
Patient 1 (non-small-cell lung cancer): Despite a very low ATP dose (=20-30 μg/kg·min), this patient spontaneously reported that he felt more energetic. Before the study, the patient was not able to independently dress, undress, get in/out of bed, to get out of a chair, or to wash his hands, face or body. After 8 weeks of ATP, the patient was able to perform all of these activities independently. Over 8 weeks, his appetite improved markedly: EORTC-QLQ-30 (4-point scale): improvement from 4 to 1, and on VAS [0-100 mm], from 13 to 63 before lunch, and from 17 to 70 before dinner. The patient's treating pulmonologist concluded a “miraculous improvement” over the 8-week treatment period. On request of the patient, ATP infusions were continued.
Patient 2 (primary liver carcinoma): This patient, who suffered from marked anorexia, spontaneously reported that he was “eating again like a building worker” after 8 weeks of ATP treatment. Indeed, appetite assessment by VAS (0-100) showed a dramatic improvement from 20 to 95 within 4 weeks. Furthermore, the patient felt less tired within 2 weeks of starting ATP infusions.
Patient 3 (melanoma): This patient reported marked improvement in appetite. This was confirmed by outcome assessment (VAS), in addition, marked amelioration in fatigue and self reliance was found. After 8 infusion, the patient pledged to continue the ATP infusions.
Patient 4 (lung cancer; study still ongoing): After 4 weeks of ATP infusions, fatigue of this patient had remarkably improved from 2.8 to 5.8 on a 7-point scale (mean of four items of SFQ). After 5 infusions, the patient decided that he felt so much better that he wanted to continue the infusions after the study.
Patient 5 (pancreas cancer; study still ongoing): After 4 infusions, the patient noted to feel much more energetic.

### Lung Cancer Patients

### Methods:

Patients with non-small-cell lung cancer, stage IIIB and IV, in the palliative treatment stage, were randomized to receive either ATP infusions (total of 10 infusions over 24 weeks) or no treatment. Outcome assessment (quality of life, blood values) was performed at regular intervals.

### Results:

In the control group, plasma lactate dehydrogenase and triglyceride concentrations increased progressively over the 24-week study period. In contrast, in the ATP group, these values remained stable throughout the study period.

Compared to the control group, the following significant and novel quality-of-life related favourable effects of ATP were found: included: reduction of dizziness, normalization of decreased sexual interest, improvement of dry mouth. Another significant: difference was the ability of patients to go shopping and to go to work.

### REFERENCES

1. Agteresch H J, Dagnelie P C, van den Berg J W, Wilson J H. Adenosine triphosphate: established and potential clinical applications. Drugs 1999; 58(2):211-32.
2. Agteresch H J, Dagnelie P C, Rietveld T, van den Berg J W, Danser A H, Wilson J H. Pharmacokinetics of intravenous ATP in cancer patients. Eur J Clin Pharmacol 2000; 56(1):49-55.
3. Leij-Halfwerk S, Agteresch H J, Sijens P E, Dagnelie P C. Adenosine triphosphate infusion increases liver energy status in advanced lung cancer patients: an in vivo 31P magnetic resonance spectroscopy study. Hepatology 2002; 35(2):421-4.
4. Rapaport E, Fontaine J. Generation of extracellular ATP in blood and its mediated inhibition of host weight loss in tumor-bearing mice. Biochem Pharmacol 1989; 38(23):4261-6.
5. Rapaport E, Fontaine J. Anticancer activities of adenine nucleotides in mice are mediated through expansion of erythrocyte ATP pools. Proc Natl Acad Sci USA 1989; 86(5):1662-6.
6. Haskell C M, Wong M, Williams A, Lee L Y. Phase I trial of extracellular adenosine 5′-triphosphate in patients with advanced cancer. Med Pediatr Oncol 1996; 27(3):165-73.
7. Haskell C M, Mendoza E, Pisters K M, Fossella F V, Figlin R A. Phase II study of intravenous adenosine 5′-triphosphate in patients with previously untreated stage IIIB and stage IV non-small cell lung cancer. Invest New Drugs 1998; 16(1):81-5.
8. Agteresch H J, Dagnelie P C, van Der Gaast A, Stijnen T, Wilson J H. Randomized clinical trial of adenosine 5′-triphosphate in patients with advanced non-small-cell lung cancer. J Natl Cancer Inst 2000; 92(4):321-328.
9. Bast A. Antioxidant Pharmacotherapy. Drugs News and Perspectives 1994; 7(8):465-472.
10. Bast A. Oxidative stress and calcium homeostasis. In: Halliwell B, O I A, editors. DNA and free radicals. New York: Ellis Horwood; 1993. p. 95-108.
11. Li N, Hao M, Phalen R F, Hinds W C, Nel A E. Particulate air pollutants and asthma. A paradigm for the role of oxidative stress in PM-induced adverse health effects. Clin Immunol 2003; 109(3):250-65.
12. Bast A, Haenen G R, Doelman C J. Oxidants and antioxidants: state of the art. Am J Med 1991; 91(3C):2S-13S.
13. Hadjigogos K. The role of free radicals in the pathogenesis of rheumatoid arthritis. Panminerva Med 2003; 45(1):7-13.
14. Richards R S, Roberts T K, McGregor N R, Dunstan R H, Butt H L. Blood parameters indicative of oxidative stress are associated with symptom expression in chronic fatigue syndrome. Redox Rep 2000; 5(1):35-41.
15. Prasad K, Lee P. Suppression of oxidative stress as a mechanism of reduction of hypercholesterolemic atherosclerosis by aspirin. J Cardiovasc Pharmacol Ther 2003; 8(1):61-9.
16. Korantzopoulos P, Papaioannides D, Galaris D, Kokkoris S. On the role of oxidative stress in accelerated atherosclerosis observed in rheumatic diseases. Joint Bone Spine 2003; 70(4):311-2.
17. Berg B M, Godbout J P, Kelley K W, Johnson R W. Alpha-tocopherol attenuates lipopolysaccharide-induced sickness behavior in mice. Brain Behav Immun 2004; 18(2):149-57.
18. van der Vliet A, Bast A. Role of reactive oxygen species in intestinal diseases. Free Radic Biol Med 1992; 12(6):499-513.
19. Gonenc A, Ozkan Y, Torun M, Simsek B. Plasma malondialdehyde (MDA) levels in breast and lung cancer patients. J Clin Pharm Ther 2001; 26(2):1414.
20. Fisher A E, Naughton D P. Why nutraceuticals do not prevent or treat Alzheimer's disease. Nutr J 2005; 4(1):14.
21. Mak S, Newton G E. The oxidative stress hypothesis of congestive heart failure: radical thoughts. Chest 2001; 120(6):2035-46.
22. Vecchiet J, Cipollone F, Falasca K, Mezzetti A, Pizzigallo E, Bucciarelli T, et al. Relationship between musculoskeletal symptoms and blood markers of oxidative stress in patients with chronic fatigue syndrome. Neurosci Lett 2003; 335(3):1514.
23. Zoroglu S S, Armutcu F, Ozen S, Gurel A, Sivasli E, Yetkin O, et al. Increased oxidative stress and altered activities of erythrocyte free radical scavenging enzymes in autism. Eur Arch Psychiatry Clin Neurosci 2004; 254(3):143-7.
24. Korantzopoulos P, Galaris D, Papaioannides D, Siogas K. The possible role of oxidative stress in heart failure and the potential of antioxidant intervention. Med Sci Monit 2003; 9(6):RA120-5.
25. Lim C S, Vaziri N D. Iron and oxidative stress in renal insufficiency. Am J Nephrol 2004; 24(6):569-75.
26. Gackowski D, Kruszewski M, Jawien A, Ciecierski M, Olinski R. Further evidence that oxidative stress may be a risk factor responsible for the development of atherosclerosis. Free Radic Biol Med 2001; 31(4):542-7.
27. Gackowski D, Banaszkiewicz Z, Rozalski R, Jawien A, Olinski R. Persistent oxidative stress in colorectal carcinoma patients. Int J Cancer 2002; 101(4):395-7.
28. Boots A W, Haenen G R, Bast A. Oxidant metabolism in chronic obstructive pulmonary disease. Eur Respir J Suppl 2003; 46:14s-27s.
29. el Moatassim C, Dornand J, Mani J C. Extracellular ATP and cell signalling. Biochim Biophys Acta 1992; 1134(1):31-45.
30. Fredholm B B. Purines and neutrophil leukocytes. Gen Pharmacol 1997; 28(3):345-50.
31. Suh B C, Kim J S, Namgung U, Ha H, Kim K T. P2X7 nucleotide receptor mediation of membrane pore formation and superoxide generation in human promyelocytes and neutrophils. J Immunol 2001; 166(11):6754-63.
32. Parvathenani L K, Tertyshnikova S, Greco C R, Roberts S B, Robertson B, Posmantur R. P2X7 mediates superoxide production in primary microglia and is up-regulated in a transgenic mouse model of Alzheimers disease. J Biol Chem 2003; 278(15): 13309-17.
33. Kuhns D B, Wright D G, Nath J, Kaplan S S, Basford R E. ATP induces transient elevations of [Ca2+]i in human neutrophils and primes these cells for enhanced O2-generation. Lab Invest 1988; 58(4):448-53.
34. Smit M J, Leurs R, Bloemers S M, Tertoolen L G, Bast A, De Laat S W, et al. Extracellular ATP elevates cytoplasmatic free Ca2+ in HeLa cells by the interaction with a 5′-nucleotide receptor. Eur J Pharmacol 1993; 247(2):223-6.
35. Goeptar A R, Haenen G R, Timmerman H, Bast A. The effects of beta-adrenergic receptor agonists on the H₂O₂ formation in alveolar macrophage suspensions are not mediated by beta-receptors. Agents Actions 1988; 25(3-4):375-7.
36. Leurs R, Timmerman H, Bast A. Inhibition of superoxide anion radical production by ebselen (PZ51) and its sulfur analogue (PZ25) in guinea pig alveolar macrophages. Biochem Int 1989; 18(2):295-9.
37. Koukourakis M I, Giatromanolaki A, Spyridis E, Bougioukas G, Didilis V, Gatter K C, et al. Lactate dehydrogenase-5 (LDH-5) overexpression in non-small-cell lung cancer tissues is linked to tumour hypoxia, angiogenic factor production and poor prognosis. Br J Cancer 2003; 89(5):877-85.
38. Ferrari S, Bacci G, Picci P, Mercuri M, Briccoli A, Pinto D, et al. Long-term follow-up and post-relapse survival in patients with non-metastatic osteosarcoma of the extremity treated with neoadjuvant chemotherapy. Ann Oncol 1997; 8(8):765-71.

1. Use of ATP for the manufacture of a medicine comprising ATP as an active ingredient for exerting a preventive or therapeutic pharmacological effect when administered to a mammal, preferably a human, selected from the group consisting of:
(a) modulating oxidative stress and the effects thereof by favourably affecting the formation or scavenging of aggressive hydroxyl radicals;
(b) modulating the inflammatory response to a strong external insult such as endotoxin (LPS) and/or phytohaemagglutinin, even under conditions of severe oxidative stress;
(c) inhibiting the inflammatory response to a strong external insult such as endotoxin (LPS) and/or phytohaemagglutinin under conditions of severe oxidative stress;
(d) exerting a local protective effect against oxidative stress in the intestine, thus preventing intestinal damage induced by several types of medication such as non-steroid anti-inflammatory drugs (NSAIDs);
(e) exerting favourable immuno-modulating and oxidative stress-reducing effects in blood from patients with oxidative stress-related disorders; and
(f) exerting favourable clinical effects in patients with different oxidative stress-related disorders such as, but not limited to, rheumatoid arthritis, intestinal disease, cancer and fatigue.

2. Use of ATP for the manufacture of a medicine comprising ATP as an active ingredient having a preventive or curative activity when administered to a mammal, preferably a human, selected from the group consisting of:
(a) tissue-protecting activity by attenuating oxidative stress under varying conditions of oxidative stress and inflammation;
(b) immune-stimulating activity by attenuating oxidative stress under varying conditions characterized by immune-incompetence or immuno-suppression, and immunomodulating activity normalizing the Th1/Th2 balance in conditions of aberrant Th1- or Th2-skewed immune response, such as auto-immune disorders and atopic diseases; and
(c) modulating and normalizing aberrant mental neurological and neuro-psychiatric states and diseases.

3. Use of ATP according to claim 1, wherein the medicine is for preventing or treating at least one of intestinal inflammatory condition, intestinal damage, and inflammatory bowel disease.

4. Use of ATP according to claim 1, wherein the medicine is for preventing or treating rheumatoid arthritis.

5. Use of ATP according to claim 1, wherein the medicine is for preventing or treating an atopic disease, including asthma.

6. Use of ATP according to claim 1, wherein the medicine is for preventing or treating a condition selected from the group consisting of fatigue, fibromyalgia, burn-out and depression.

7. Use of ATP according to claim 1, wherein the medicine is for preventing or treating a disease or disorder or condition selected from the group consisting of cancer during and after treatment by at least one of surgery, radiotherapy and chemotherapy, neurological and mental diseases/conditions, and another condition of an elevated or aberrant inflammatory response.

8. A method of preventing or treating an individual for a disease or disorder or condition selected from the group consisting of intestinal inflammation, intestinal damage, rheumatoid arthritis, COPD, cancer during or after treatment by at least one of surgery, radiotherapy, and chemotherapy, a neurological or mental disorder, an atopic disease including asthma, and another condition of elevated or aberrant inflammatory response, which comprises administering to said individual in need thereof a medicine comprising an effective amount of ATP.

9. Use of ATP according to claim 1, wherein the medicine is in the form of a pharmaceutical composition or a nutritional composition.

10. Use of ATP according to claim 9, wherein the medicine is in a lyophilized form.

11. A method according to claim 8, wherein the medicine is in the form of a pharmaceutical composition or a nutritional composition.

12. A method according to claim 11, wherein the medicine is in a lyophilized form.

13. Use of ATP according to claim 2, wherein the medicine is for preventing or treating at least one of intestinal inflammatory condition, intestinal damage, and inflammatory bowel disease.

14. Use of ATP according to claim 2, wherein the medicine is for preventing or treating rheumatoid arthritis.

15. Use of ATP according to claim 2, wherein the medicine is for preventing or treating an atopic disease, including asthma.

16. Use of ATP according to claim 2, wherein the medicine is for preventing or treating a condition selected from the group consisting of fatigue, fibromyalgia, burn-out and depression.

17. Use of ATP according to claim 2, wherein the medicine is for preventing or treating a disease or disorder or condition selected from the group consisting of cancer during and after treatment by at least one of surgery, radiotherapy and chemotherapy, neurological and mental diseases/conditions, and another condition of an elevated or aberrant inflammatory response.
